SEQUENCE LISTING (24 sequences)

SEQ ID NO: 1 - DNA, 240 bp, Elaeis guineensis
  misc_feature (1)..(240): Elaeis guineensis linkage group LG2 1516571 to 1516810
cgttggagcaccgtacccaccaccgccattaccagcctctaagcaagcagaggagatgatcctgtccatgcacaccatgaaatctccaaccatctttctccctcttccgggtgtctgctgttgccgtgctctgtttcggacggaggaacagaggaaaatggcaggggatggggaggtttagaaacgagaggaataaaagagaagaggaccagcctgggcgcgaaactactaaaatgcccc

SEQ ID NO: 2 - DNA, 240 bp, Elaeis guineensis
  misc_feature (1)..(240): Elaeis guineensis linkage group LG2 4215587 to 4215826
aaagtgaaaataagtaccacaaaagattatatgtggccaaaggcgataattatagtttgtcaaagtaacacaatgtaataaggaaggaagaactaaccctaaacaagatatatggacaactttttatgcaaatatgtaataagacccttcatgaaccactgaccttatgtagggtgcataaaaagccagtctggtgctcattgaaggtacgctatgtgatacaatgcgaagcaatacttc

SEQ ID NO: 3 - DNA, 240 bp, Elaeis guineensis
  misc_feature (1)..(240): Elaeis guineensis linkage group LG2 4858549 to 4858788
cacatagatctagcttatttcagcattatatgatcatgggatgggtggttatacccaccactaatcatgttctgtgctttctgcactggtggtttctgatatgccgactctatatcgatagtctgaatatgtcgactatatgttggtaattgtgaaagttagaatgaccatcggtcggtaattctgatatgccgatggtcatctgatatgccgatggtcatcagtttgaaaccacttaag

SEQ ID NO: 4 - DNA, 240 bp, Elaeis guineensis
  misc_feature (1)..(240): Elaeis guineensis linkage group LG2 5594023 to 5594262
ggctactgtttttatctttttttccattccatcattcgctacttaatttataattgaaataaactaaatacgaagagttttatcatctgtcaatttttaattgaaatcagccaaatactaagagcgtttaggcttatctgctttattggaagaacaacgaagctcgcaatcacaccaaaactagagcaaagaagaagaagagaagaagaactgaggagagcatggaagagagagcaagaa

SEQ ID NO: 5 - DNA, 240 bp, Elaeis guineensis
  misc_feature (1)..(240): Elaeis guineensis linkage group LG2 33949264 to 33949503
tgctaactgaagaagacgtaccaactgcaccagaacctgctccaaccactgaagtcgtatcagagcaaggcgatgaggaagccgactggtgatcgatgcaacacttcttctttttgtagtaggattttgtaatcggacctttggtctaattttgtaatcttttcttcaatgaatgaaaaaaaattttaagtaccgaatctctctttgtatttgtacttttaatgtcattgaataatagtt

SEQ ID NO: 6 - DNA, 240 bp, Elaeis guineensis
  misc_feature (1)..(240): Elaeis guineensis linkage group LG2 34109865 to 34110104
acttagagggaaataagctgcaatttggagttaatcaatcttcccaatcctcgagggacggggaagcgttccgggggctgggagagagctaactctatgagcaaattagttgttagttagctaatcatgcaaagccaagagattattgagtgatattaaccatagaaactataagcatgatacatcaatagagtggatggaaaactaaagacaactcgctcgagataagaaatgtaaatg

SEQ ID NO: 7 - DNA, 240 bp, Elaeis guineensis
  misc_feature (1)..(240): Elaeis guineensis linkage group LG3 43405853 to 43406092
tgggtccaacttaaattctcataagaacacgaaacagatgcaatccagagaataatacatgcaaagatatttgggttgcattttttataaaaagaataattaattttttatcgcgatatatactgttagatcttgttttgcatttagctcaaagcaatcggaactctctttcagatgcttgcatggatcttatagtaaatattgtgtgattgaacagtacatatcacgagaagaagatac

SEQ ID NO: 8 - DNA, 240 bp, Elaeis guineensis
  misc_feature (1)..(240): Elaeis guineensis linkage group LG3 43834027 to 43834266
agatggtggtacctttctcattagagaggttgcagcatcctttctcccagcccttataaccttcctccaagcatggttggaagggccaatttcctttgtttccttattggatccttggagctttccaatgcaatgggagttgccgaggaagaaagctctataagtctttaccctcttctattcaaggaggaggtgatagtttgagagcttaaagaaatcaaggctccccttacttcgagc

SEQ ID NO: 9 - DNA, 240 bp, Elaeis guineensis
  misc_feature (1)..(240): Elaeis guineensis linkage group LG3 44126148 to 44126387
ttgacttattcaaaatatctcctagattgcatcatgagctccagacggccagcttgaaaacttgttcaccgatgccgacatcaagcattggatgatgtcgataaccctacgagatactccaggctctctaaacgtgctgtaaaattaattttgaaaaataaatattaactctaaaatagctaaattttaaacttattaattgaagttgtagtacttacgagtagctctaaacgtgaatga

SEQ ID NO: 10 - DNA, 240 bp, Elaeis guineensis
  misc_feature (1)..(240): Elaeis guineensis linkage group LG3 44192858 to 44193097
attcccagccaagctttctgatttttccacaaaatgaaaacctttctcactcaaagaagctgaaatctttttttcttctcaagataatagattggcttgaaagagatacctttgaagaaggtgtggagagaaaaggagtcgaagctgactaatctttgtccttatgagctgagctttttagttgccttcttagagaagcagatgctggctcaaccacaatcattatgggactcgaagact

SEQ ID NO: 11 - DNA, 240 bp, Elaeis guineensis
  misc_feature (1)..(240): Elaeis guineensis linkage group LG4 30702027 to 30702266
ttcatgatagtctctatgtctatcatcatttgtcattccctttagcaaaacattgtccctgactgggtcaagctttgatttcttcaagacggtaagagacattgtgatttgcttgcaattggcaggacaggtttcatgcggggtctctgttttgcctgaattagcatggccagtaatccatgccaattttatagggctaatgtcaagcttataatgggtcataaaatctgcttagtcctc

SEQ ID NO: 12 - DNA, 240 bp, Elaeis guineensis
  misc_feature (1)..(240): Elaeis guineensis linkage group LG4 31148391 to 31148630
gatcacttatttctctcatgcaactattcagctcaagtctggcagtacttccctaatcgtttcaacatcaggagactgctacatttacttccgggatcactactttcacctggactaaagagtccgttcgcacttacagctgggataggagagcctcctgatgacttgtatttggcccatgaggaaaaaacaccaaaatctagtctaaaagctcttgccaactgcacttggactacatta

SEQ ID NO: 13 - DNA, 240 bp, Elaeis guineensis
  misc_feature (1)..(240): Elaeis guineensis linkage group LG4 33166529 to 33166768
gattcatgggcatgatcgatcgaaggtatttatctgaccgatgttgctccatctctcacaatttaattatattattaatttgacataagaggtagcactcgaaggtaacactggaagattggtgaaatgatatatggagaaatatgaggatcagtttagatatggatttaattccatatcactgacaccagaaatattttaggtacttttttatgaggtgagattttaaatcattacaaa

SEQ ID NO: 14 - DNA, 240 bp, Elaeis guineensis
  misc_feature (1)..(240): Elaeis guineensis linkage group LG4 33451315 to 33451554
tttaaatttgtaattgcataagtaccataactaaaaattatgatagacaagtgttactaaagaacattacttatttcataattacaccatgaatgagaaaagaaagtccctgttatggacttatatagcacttgcagaacttcacagagcaaatcagtcagatatcatatagcaagatcaaaatgaacaggtcaggatgttataaagctgcaaaattgattctatgaattgaaaaagaga

SEQ ID NO: 15 - DNA, 240 bp, Elaeis guineensis
  misc_feature (1)..(240): Elaeis guineensis linkage group LG7 35906266 to 35906505
tgagggaagaatgattgatttgcctaacctctttgaatctgagtcaccttgataaatagcatccatcaaaccattataaagatctacttttagctttgtctgattatttcttatccagcagaatctttcttcttcaattaccatatagcagtttactatgaattgttggaataatctgcctgcttgatgaagtgtgtgggctttattcagtctttgttgaagtctaaaacaataatgtta

SEQ ID NO: 16 - DNA, 240 bp, Elaeis guineensis
  misc_feature (1)..(240): Elaeis guineensis linkage group LG7 36257469 to 36257708
tgtgataggcatagggattgctgcaactgctgtggggctattggttgggggaatcgctgccgcggtgactcggaagaagtgatgagttcgctgacaaatacaacatcatgaatgatttagatgctgctgtttcttgtaatgtatttagctgcttaagtgtctttacttgaatattgtgttatttctaatcactatatgtgcgtaagaaccaagagtctaagctttgcagggatgtatttt

SEQ ID NO: 17 - DNA, 240 bp, Elaeis guineensis
  misc_feature (1)..(240): Elaeis guineensis linkage group LG10 29233675 to 29233914
agatcaacaacacttcacaaagcttcaagaacatagtatgaattttgaattttatacaaggactcaagctatgtggagccataatatgtatgcattgaacaagtttttttcgattcaactttcagtgtgaaataacctgctatgaccacaggacaatataaaatgcaagcataattaataaaaaattattacttatatgaaggtgtattctacaagtttgtatacaaatgcctaatcatg

SEQ ID NO: 18 - DNA, 240 bp, Elaeis guineensis
  misc_feature (1)..(240): Elaeis guineensis linkage group LG10 29611963 to 29612202
tgaaaccaagccaaaattgaggctcctttctcagttattctcatgtactagctaactcacaggttgttgggactttggcataggtcaaaggacaaagtaggcaaggaatcggggactttggcgatgaacagcgatggtgaacatggtcagaaaaagggatagagataaaaaaaataagaaatttttttttcttgatttttcagcaaaaccaacggtttatggttgtgcctaaggctatag

SEQ ID NO: 19 - DNA, 240 bp, Elaeis guineensis
  misc_feature (1)..(240): Elaeis guineensis linkage group LG11 13470988 to 13471227
cggactggactgaggcttcctctccaccccttctttacaactctcctccagtatcttgagatgttgccatgttcgatagtttcaaactcatggtgacatatctatggctttacaatagtatgtgtgctggctgggattggtcctagtatcatgctcttcttttctttttttagcctcaagaggcattcggatttgcacgagtggtggtatatgatgacacaaaggaagaagaatccactg

SEQ ID NO: 20 - DNA, 240 bp, Elaeis guineensis
  misc_feature (1)..(240): Elaeis guineensis linkage group LG11 13734477 to 13734716
acttagttgctaatcctgtgtttcatgcccgcactaagtacattgagatacaatattactttatgcaggaacaagttgctaccaagcgtctttaggtgtccttcatctccaataaggaccagctcgccaacatcttcaccaagcaattgagccggcagtggtttactctcatccaagacaagctacaagtgatcgaacctcccttgtgcttgaggaagagtattaaaggaagcaaccatg

SEQ ID NO: 21 - DNA, 240 bp, Elaeis guineensis
  misc_feature (1)..(240): Elaeis guineensis linkage group LG13 24620951 to 24621190
taccattgttaagatttggcacccactttcacttcacactcacattctagcctacgttagaagaataatttgtcctaaaagccatgtttagcacctatagctttgcctaaatgcatggaaaaactaagctagatcaaaatttggatagaagctaagccacttgcataattgacttaaatgtccaaccttgaagaaagctgagacgtagtgagagaagcaaggatgctaaacgggtgtcac

SEQ ID NO: 22 - DNA, 240 bp, Elaeis guineensis
  misc_feature (1)..(240): Elaeis guineensis linkage group LG13 24988766 to 24989005
acaatgaggggcatgatcaacccgagcctgatcagaccccgagcttgatcaagctcgggtctgatcaagcttggtctgtaattaatttcaagcttgaattttgaaatcaagcttgatttgtttgtcattactttgagctcagtccaagattaatgtcaaagcaagtggaatataagcccaattaagcagctcgataatcctaaagtgagctgaaaccagttcaagcttggttcaagcttg

SEQ ID NO: 23 - DNA, 240 bp, Elaeis guineensis
  misc_feature (1)..(240): Elaeis guineensis linkage group LG15 6941783 to 6942022
aaggtcatgatttacatgatgaagacttcggatgtcattcccgaactgacttctcggctatattcatgctatgtcatttctgtcagttaattcgacttcgatcattatcatcggattattttcaaattttttttttgtgaaagatcctaacttgtgtaaggatgtccttctattgctaacattccgtcaataagtattcagagatctcaattttttagtcaaaatatctctaaagaatca

SEQ ID NO: 24 - DNA, 240 bp, Elaeis guineensis
  misc_feature (1)..(240): Elaeis guineensis linkage group LG15 7160303 to 7160542
gttattataagtttatataggttgaaatcaaggaattcgaatacatttgcattttagccaaatggatcaaataaggcaaagttaacctaagcctactctaaatttattttagctttagattttaaaataaagcccaacagaaatgctctacaatttttatttctagctgattagtcttttgcttaactttttttaaaagacaaaaagaaaaaatgattcacatcttgcaaatccaattgc

(19) United States
(12) Patent Application Publication
(10) Pub. No.: US 2018/0274016 A1
(43) Pub. Date: Sep. 27, 2018

(54) METHODS FOR PREDICTING PALM OIL YIELD OF A TEST OIL PALM PLANT

(71) Applicant: SIME DARBY PLANTATION INTELLECTUAL PROPERTY SDN. BHD., Petaling Jaya, Selangor (MY)

(72) Inventors: Ai Ling Ong, Serdang (MY); Qi Bin Kwong, Serdang (MY); Chee Keng Teh, Banting (MY); Mohamed Mohaimi, Banting (MY); Fook Tim Chew, National University of Singapore (SG); David Ross Appleton, Serdang (MY); Harikrishna Kulaveerasingam, Carey Island (MY)

(21) Appl. No.: 15/767,644

(22) PCT Filed: Nov. 9, 2016
(86) PCT No.: PCT/MY2016/000076

§ 371 Date: Apr. 11, 2018

(30) Foreign Application Priority Data
Dec. 30, 2015 (MY) PI 2015003079

(51) Int. Cl. C12Q 1/6827; C12Q 1/6895

(52) U.S. Cl. CPC C12Q 1/6827; C12Q 2600/13; C12Q 2600/156; C12Q 1/6895

(57) ABSTRACT

Methods for predicting palm oil yield of a test oil palm plant are disclosed. The methods comprise determining, from a sample of a test oil palm plant of a population, at least a first SNP genotype, corresponding to a first SNP marker, located in a first QTL for a high-oil-production trait and associated, after stratification and kinship correction, with the high-oil-production trait with a genome-wide −log₁₀(p-value) of at least 7.0 in the population or having a linkage disequilibrium r² value of at least 0.2 with respect to a first other SNP marker linked thereto and associated, after stratification and kinship correction, with the high-oil-production trait with a genome-wide −log₁₀(p-value) of at least 7.0 in the population. The methods also comprise comparing the first SNP genotype to a corresponding first reference SNP genotype and predicting palm oil yield of the test plant based on extent of matching of the SNP genotypes.

### TECHNICAL FIELD

This application relates to methods for predicting palm oil yield of a test oil palm plant, and more particularly to methods for predicting palm oil yield of a test oil palm plant comprising determining, from a sample of a test oil palm plant of a population of oil palm plants, at least a first single nucleotide polymorphism (also termed SNP) genotype of the test oil palm plant, the first SNP genotype corresponding to a first SNP marker, comparing the first SNP genotype of the test oil palm plant to a corresponding first reference SNP genotype indicative of the high-oil-production trait in the same genetic background as the population, and predicting palm oil yield of the test oil palm plant based on the extent to which the first SNP genotype of the test oil palm plant matches the corresponding first reference SNP genotype.

### BACKGROUND ART

The African oil palm Elaeis guineensis Jacq. is an important oil-food crop. Oil palm plants are monoecious, i.e. single plants produce both male and female flowers, and are characterized by alternating series of male and female inflorescences. The male inflorescence is made up of numerous spikelets, and can bear well over 100,000 flowers. Oil palm is naturally cross-pollinated by insects and wind. The female inflorescence is a spadix which contains several thousands of flowers borne on thorny spikelets. A bunch carries 500 to 4,000 fruits. The oil palm fruit is a sessile drupe that is spherical to ovoid or elongated in shape and is composed of an exocarp, a mesocarp containing palm oil, and an endocarp surrounding a kernel.

Oil palm is important both because of its high yield and because of the high quality of its oil. Regarding yield, oil palm is the highest yielding oil-food crop, with a recent average yield of 3.67 tonnes per hectare per year and with best progenies known to produce about 10 tonnes per hectare per year. Oil palm is also the most efficient plant known for harnessing the energy of sunlight for producing oil. Regarding quality, oil palm is cultivated for both palm oil, which is produced in the mesocarp, and palm kernel oil, which is produced in the kernel. Palm oil in particular is a balanced oil, having almost equal proportions of saturated fatty acids (≈55% including 45% of palmitic acid) and unsaturated fatty acids (≈45%), and it includes beta carotene. The palm kernel oil is more saturated than the mesocarp oil. Both are low in free fatty acids. The current combined output of palm oil and palm kernel oil is about 50 million tonnes per year, and demand is expected to increase substantially in the future with increasing global population and per capita consumption of oils and fats.

Although oil palm is the highest yielding oil-food crop, current oil palm crops produce well below their theoretical maximum, suggesting potential for improving yields of palm oil through improved selection and identification of high yielding oil palm plants. Conventional methods for identifying potential high-yielding oil palms, for use in crosses to generate progeny with higher yields as well as for commercial production of palm oil, require cultivation of oil palms and measurement of production of palm oil thereby over the course of many years, though, which is both time and labor intensive. Moreover, the conventional methods are based on direct measurement of palm oil content of sampled fruits, and thus result in destruction of the sampled fruits. In addition, conventional breeding techniques for propagation of oil palm for palm oil production are also time and labor intensive, particularly because the most productive, and thus commercially relevant, oil palms exhibit a hybrid phenotype with respect to fruit type, based on heterozygosity with respect to a gene termed SHELL, i.e. having one wild-type allele of SHELL (sh+) and one mutant allele of SHELL (sh−), which makes propagation thereof by direct hybrid crosses impractical.

Quantitative trait loci (also termed QTL) marker programs based on linkage analysis have been implemented in oil palm with the aim of improving upon conventional breeding techniques, as taught for example by Billotte et al., Theoretical & Applied Genetics 120:1673-1687 (2010). Linkage analysis is based on recombination observed in a family within recent generations and often identifies poorly localized QTLs for complex phenotypes, though, and thus large families are needed for better detection and confirmation of QTLs, limiting practicality of this approach for oil palm.

QTL marker programs based on association analysis for the purpose of identifying candidate genes may be a possibility for oil palm too, as discussed for example by Ong et al., WO2014/129885, with respect to plant height. A focus on identifying candidate genes may be of limited benefit in the context of traits that are determined by multiple genes though, particularly genes that exhibit low penetrance with respect to the trait.

QTL marker programs based on genome-wide association studies have been carried out in human and rice, among others, as taught by Hirota et al., Nature Genetics 44:1222-1226 (2012), and Huang et al., Nature Genetics 42961-967 (2010), respectively. Application of this approach to oil palm has not been practical, though, because commercial oil palms tend to be generated from genetically narrow breeding materials.

Recent advances have been made in predicting whether individual oil palm plants will exhibit the hybrid phenotype with respect to the SHELL gene based on determining genotypes of oil palm plants, as taught by Singh et al., WO2013/142187 and Singh et al., WO2015/010008. This may allow for a reduction of resources expended in cultivating oil palm plants that will not exhibit the hybrid phenotype, for purposes of commercial production of palm oil, but would not be expected to provide a basis for increasing palm oil yield among oil palm plants expressing the hybrid phenotype themselves.

Recent advances also have been made in identifying binding partners of the product of the SHELL gene, based on binding of the product of the SHELL gene to SEPALLATA (SEP) orthologs from rice (Oryza sativa) in a yeast two-hybrid system, and identification of numerous genes encoding potential SEP-like proteins in oil palm, as taught by Singh et al., WO2015/010131. It remains to be determined, though, whether and to what extent SEP-like proteins play a role in modulating morphology of oil palm fruit and/or palm oil yield.

Accordingly, a need exists to improve oil palm through improved methods for predicting palm oil yields of oil palm plants.

### DISCLOSURE OF INVENTION

A method for predicting palm oil yield of a test oil palm plant is disclosed. The method comprises a step of (i) determining, from a sample of a test oil palm plant of a population of oil palm plants, at least a first single nucleotide polymorphism (SNP) genotype of the test oil palm plant. The first SNP genotype corresponds to a first SNP marker. The first SNP marker is located in a first quantitative trait locus (QTL) for a high-oil-production trait. The first SNP marker also is associated, after stratification and kinship correction, with the high-oil-production trait with a genome-wide −log₁₀(p-value) of at least 7.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 7.0 in the population. The method also comprises a step of (ii) comparing the first SNP genotype of the test oil palm plant to a corresponding first reference SNP genotype indicative of the high-oil-production trait in the same genetic background as the population. The method also comprises a step of (iii) predicting palm oil yield of the test oil palm plant based on the extent to which the first SNP genotype of the test oil palm plant matches the corresponding first reference SNP genotype. The first QTL is a region of the oil palm genome corresponding to one of:

(1) QTL region 1, extending from nucleotide 1516571 to 4215826 of chromosome 2;
(2) QTL region 2, extending from nucleotide 4858549 to 5594262 of chromosome 2;
(3) QTL region 3, extending from nucleotide 33949264 to 34110104 of chromosome 2;
(4) QTL region 4, extending from nucleotide 43405853 to 43834266 of chromosome 3;
(5) QTL region 5, extending from nucleotide 44126148 to 44193097 of chromosome 3;
(6) QTL region 6, extending from nucleotide 30702027 to 31148630 of chromosome 4;
(7) QTL region 7, extending from nucleotide 33166529 to 33451554 of chromosome 4;
(8) QTL region 8, extending from nucleotide 35906266 to 36257708 of chromosome 7;
(9) QTL region 9, extending from nucleotide 29233675 to 29612202 of chromosome 10;
(10) QTL region 10, extending from nucleotide 13470988 to 13734716 of chromosome 11;
(11) QTL region 11, extending from nucleotide 24620951 to 24989005 of chromosome 13; or
(12) QTL region 12, extending from nucleotide 6941783 to 7160542 of chromosome 15.

### BRIEF DESCRIPTION OF DRAWINGS

FIG. 1 shows quantile-quantile (Q-Q) plots of observed −log₁₀(p-values) versus expected −log₁₀(p-values) for GWAS, based on a compressed mixed linear model (also termed MLM), in 27 oil palm origins as discussed below, for (A) shell-to-fruit and (B) mesocarp-to-fruit.

FIG. 2 shows Manhattan plots, based on a compressed mixed linear model (also termed MLM), in 27 oil palm origins as discussed below, for (A) shell-to-fruit (also termed S/F) and (B) mesocarp-to-fruit (also termed M/F).

FIG. 3 is an illustration of an approach for defining a range of a QTL region according to a linkage disequilibrium r² value of at least 0.2 as threshold, wherein the highlighted range (i.e. SNP A to SNP D, as enclosed in open rectangle) is the selected QTL region in accordance with the method of predicting palm oil yield of a test oil palm plant.

FIG. 4 is a plot of prediction accuracy (y-axis) versus number of QTLs represented in analysis (x-axis) in 27 oil palm origins as discussed below for shell-to-fruit (also termed S/F).

FIG. 5 is a plot of prediction accuracy (y-axis) versus number of QTLs represented in analysis (x-axis) in 27 oil palm origins as discussed below for mesocarp-to-fruit (also termed M/F).

### BEST MODE FOR CARRYING OUT THE INVENTION

The application is drawn to methods for predicting palm oil yield of a test oil palm plant. The methods comprise steps of (i) determining, from a sample of a test oil palm plant of a population of oil palm plants, at least a first single nucleotide polymorphism (SNP) genotype of the test oil palm plant, (ii) comparing the first SNP genotype of the test oil palm plant to a corresponding first reference SNP genotype indicative of the high-oil-production trait in the same genetic background as the population, and (iii) predicting palm oil yield of the test oil palm plant based on the extent to which the first SNP genotype of the test oil palm plant matches the corresponding first reference SNP genotype. The first SNP genotype corresponds to a first SNP marker. The first SNP marker is located in a first quantitative trait locus (QTL) for a high-oil-production trait. The first SNP marker also is associated, after stratification and kinship correction, with the high-oil-production trait with a genome-wide −log₁₀(p-value) of at least 7.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 7.0 in the population. The first QTL is a region of the oil palm genome corresponding to one of QTL regions 1 to 12, as described in more detail below.

By conducting genome resequencing and genome-wide association studies of 4,623 oil palm plants of 27 oil palm origins, including a commercially relevant Ulu Remis dura×AVROS pisifera population and a commercially relevant Banting dura×AVROS pisifera population, among others, and by including application of stratification and kinship correction, it has been determined that SNP markers that are located in 12 QTL regions of the oil palm genome and that are associated, after stratification and kinship correction, with a high-oil-production trait can be used to achieve correlation accuracies of, for example, 0.32 and 0.30, respectively, regarding the high-oil-production traits shell-to-fruit and mesocarp-to-fruit.

Without wishing to be bound by theory, it is believed that identification of the 12 QTL regions and SNP markers therein that are associated, after stratification and kinship correction, with the high-oil-production trait will enable more rapid and efficient selection of candidate agricultural production palms and candidate breeding palms, from among the 27 oil palm origins and others. Stratification and kinship correction reduce false-positive signals due to recent common ancestry of small groups of individuals within the population of oil palm plants from which a test oil palm plant is sampled, thereby making practical the method for predicting palm oil yield of a test oil palm plant based on association. The methods will enable identification of potential high-yielding palms, for use in crosses to generate progeny with higher yields and for commercial production of palm oil, without need for cultivation of the palms to maturity, thus bypassing the need for the time and labor intensive cultivations and measurements, the destructive sampling of fruits, and the impracticality of direct hybrid crosses that are characteristic of conventional approaches. For example, the methods can be used to choose oil palm plants for germination, cultivation in a nursery, cultivation for commercial production of palm oil, cultivation for further propagation, etc., well before direct measurement of palm oil production by the test oil palm plant could be accomplished. Also for example, the methods can be used to accomplish prediction of palm oil yields with greater efficiency and/or less variability than by direct measurement of palm oil production. The methods can be used advantageously with respect to even a single SNP, given that improvements in palm oil yield that seem small on a percentage basis still can have a dramatic effect on overall palm oil yields, given the large scale of commercial cultivations. The methods also can be used advantageously with respect to combinations of two or more SNPs, e.g. a first SNP genotype and a second SNP genotype, or a first SNP genotype to a twelfth SNP genotype, given additive and/or synergistic effects.

The terms “high-oil-production trait,” “high yield,” “high-yielding,” and “oil yield,” as used with respect to the methods disclosed herein, refer to yields of palm oil in mesocarp tissue of fruits of oil palm plants.

The singular forms “a,” “an,” and “the” are intended to include the plural forms as well, unless the context clearly indicates otherwise.

As noted above, a method for predicting palm oil yield of a test oil palm plant is disclosed. The method comprises a step of (i) determining, from a sample of a test oil palm plant of a population of oil palm plants, at least a first SNP genotype of the test oil palm plant.

The SNP genotype of the test oil palm plant corresponds to the constitution of SNP alleles at a particular locus, or position, on each chromosome in which the locus occurs in the genome of the test oil palm plant. A SNP is a polymorphic variation with respect to a single nucleotide that occurs at such a locus on a chromosome. A SNP allele is the specific nucleotide present at the locus on the chromosome. For oil palm plants, which are diploid and which thus inherit one set of maternally derived chromosomes and one set of paternally derived chromosomes, the SNP genotype corresponds to two SNP alleles, one at the particular locus on the maternally derived chromosome and the other at the particular locus on the paternally derived chromosome. Each SNP allele may be classified, for example, based on allele frequency, e.g. as a major allele (A) or a minor allele (a). Thus, for example, the SNP genotype can correspond to two major alleles (A/A), one major allele and one minor allele (A/a), or two minor alleles (a/a).

The test oil palm plant can be an oil palm plant corresponding to an important oil-food crop. For example, the test oil palm plant can correspond to African oil palm Elaeis guineensis.

The test oil palm plant can be an oil palm plant in any suitable form. For example, the test oil palm plant can be a seed, a seedling, a nursery phase plant, an immature phase plant, a cell culture plant, a zygotic embryo culture plant, or a somatic tissue culture plant. Also for example, the test oil palm plant can be a production phase plant, a mature palm, a mature mother palm, or a mature pollen donor.

A test oil palm plant in the form of a seed, a seedling, a nursery phase plant, an immature phase plant, a cell culture plant, a zygotic embryo culture plant, or a somatic tissue culture plant is in a form that is not yet mature, and thus that is not yet producing palm oil in amounts typical of commercial production, if at all. Accordingly, the method as applied to a test oil palm plant in such a form can be used to predict palm oil yield of the test oil palm plant before the test oil palm plant has matured sufficiently to allow direct measurement of palm oil production by the test oil palm plant during commercial production.

A test oil palm plant in the form of a production phase plant, a mature palm, a mature mother palm, or a mature pollen donor is in a form that is mature. Accordingly, the method as applied to a test oil palm plant in such a form can be used to predict palm oil yield of the test oil palm as an alternative to direct measurement of palm oil yield.

The population of oil palm plants from which the test oil palm plant is sampled can comprise any suitable population of oil palm plants. The population can be specified in terms of fruit type and/or identity of the breeding material from which the population was generated.

In this regard, fruit type is a monogenic trait in oil palm that is important with respect to breeding and commercial production. Oil palms with either of two distinct fruit types are generally used in breeding and seed production through crossing in order to generate palms for commercial production of palm oil, also termed commercial planting materials or agricultural production plants. The first fruit type is dura (genotype: sh+ sh+), which is characterized by a thick shell (also termed seed coat) corresponding to 28% to 35% of the fruit by weight, with no ring of black fibres around the kernel of the fruit. For dura fruits, the ratio of mesocarp to fruit varies from 50% to 60%, with extractable oil content in proportion to bunch weight of 18% to 24%. The second fruit type is pisifera (genotype: sh− sh−), which is characterized by the absence of a shell, the vestiges of which are represented by a ring of fibres around a small kernel. Accordingly, for pisifera fruits, the ratio of mesocarp to fruit is 90% to 100%. The ratio of mesocarp oil to bunch is comparable to the dura at 16% to 28%. Pisiferas are however usually female sterile as the majority of bunches abort at an early stage of development.

Crossing dura and pisifera gives rise to palms with a third fruit type, the tenera (genotype: sh+ sh−). Tenera fruits have thin shells, typically corresponding to 8% to 10% of the fruit by weight, corresponding to a thickness of 0.5 to 4 mm, around which is a characteristic ring of black fibres. For tenera fruits, the ratio of mesocarp to fruit is comparatively high, typically in the range of 60% to 80%. Commercial tenera palms generally produce more fruit bunches than duras, although mean bunch weight is lower. The ratio of mesocarp oil to bunch is in the range of 20% to 30%, the highest of the three fruit types, and thus tenera are typically used as commercial planting materials.

Identity of the breeding material can be based on the source and breeding history of the breeding material. Dura palm breeding populations used in Southeast Asia include Serdang Avenue, Ulu Remis (which incorporated some Serdang Avenue material), Banting, Johor Labis, and Elmina estate, including Deli Dumpy, all of which are derived from Deli dura. Pisifera breeding populations used for seed production are generally grouped as Yangambi, AVROS, Binga, and URT. Other dura and pisifera populations are used in Africa and South America.

Oil palm plantation/breeding programs in Southeast Asia are using Deli dura origin, which originated from the four famous dura palms at Bogor in the year 1848. The Deli dura materials were subsequently distributed to several research stations across the region. Each station focused on different selection preferences over generations, leading to some differentiation between subpopulations, termed breeding populations of restricted origin (also termed BPRO). The important breeding populations of restricted origin derived from Deli dura are Ulu Remis (also termed UR) and Johor Labis (also termed JL). The Ulu Remis origin was selected for high bunch number and high sex ratio (defined as ratio of females to total inflorescences) in Marihat Baris, Sumatra. Instead of bunch number, Socfindo in Sumatra had developed Johor Labis origin for bigger bunches (high bunch weight) and thinner shells.

Dura palms were commercially planted in Southeast Asia before the 1960's. The Banting dura (also termed BD) was discovered in the commercial Deli dura planted in 1958 in Dusun Durian Estate. The material was selected for good bunch traits and number. Banting dura has become an important maternal source.

African dura materials are inferior to Deli dura. To increase oil yield, the main planting materials in Africa were tenera (dura×pisifera). This provided an opportunity to discover a superior pollen source, i.e. AVROS pisifera. The material originated from the renowned Djongo palms that were planted in Eala Botanical Garden in Yangambi, Zaire, now the Democratic Republic of the Congo. The material was then further selected and produced BM119 at Kelanang Bharu Division of Dusun Durian Estate. The AVROS pisifera confers superiority in growth uniformity, general combining ability, precocity, and mesocarp oil yield in Deli×AVROS progeny (tenera). Thus, the introduction of Deli dura×BM119 AVROS pisifera in the region resulted in an increase in oil per hectare of 30% since the 1960's.

Oil palm breeding is primarily aimed at selecting for improved parental dura and pisifera breeding stock palms for production of superior tenera commercial planting materials. Such materials are largely in the form of seeds although the use of tissue culture for propagation of clones continues to be developed. Generally, parental dura breeding populations are generated by crossing among selected dura palms. Based on the monogenic inheritance of fruit type, 100% of the resulting palms will be duras. After several years of yield recording and confirmation of bunch and fruit characteristics, duras are selected for breeding based on phenotype. In contrast, pisifera palms are normally female sterile and thus breeding populations thereof must be generated by crossing among selected teneras or by crossing selected teneras with selected pisiferas. The tenera×tenera cross will generate 25% duras, 50% teneras, and 25% pisiferas. The tenera×pisifera cross will generate 50% teneras and 50% pisiferas. The yield potential of pisiferas is then determined indirectly by progeny testing with the elite duras, i.e. by crossing duras and pisiferas to generate teneras, and then determining yield phenotypes of the fruits of the teneras over time. From this, pisiferas with good general combining ability are selected based on the performance of their tenera progenies. Intercrossing among selected parents is also carried out with progenies being carried forward to the next breeding cycle. This allows introduction of new genes into the breeding programme to increase genetic variability.

Oil palm cultivation for commercial production of palm oil can be improved by use of the superior tenera commercial planting materials. Priority selection objectives include high oil yield per unit area in terms of high fresh fruit bunch yield (also termed FFB) and high oil to bunch ratio (also termed O/B) (thin shell, thick mesocarp), high early yield (precocity), and good oil qualities, among other traits. Progeny plants may be cultivated by conventional approaches, e.g. seedlings may be cultivated in polyethylene bags in pre-nursery and nursery settings, raised for about 12 months, and then planted as seedlings, with progeny that are known or predicted to exhibit high yields chosen for further cultivation, among other approaches.

As mentioned above, by conducting genome resequencing and genome-wide association studies of 4,623 oil palm plants of 27 oil palm origins, including application of stratification and kinship correction, it has been determined that SNP markers that are located in 12 QTL regions of the oil palm genome and that are associated, after stratification and kinship correction, with a high-oil-production trait can be used to achieve correlation accuracies of, for example, 0.32 and 0.30, respectively, regarding the high-oil-production traits shell-to-fruit and mesocarp-to-fruit. The 27 oil palm origins include the following: (1) (Banting dura (also termed BD)×Nigerian Institute for Oil Palm Research (also termed NIFOR))×Jenderata, (2) Deli×Algemene Vereniging van Rubberplanters ter Oostkust van Sumatra (also termed AVROS), (3) Deli×Ekona, (4) (Elaeis guineensis×Elaeis oleifera) hybrid×AVROS, (5) Ekona×AVROS, (6) Gunung Melayu (also termed GM)×Dumpy AVROS (also termed DA), (7) Johor Labis (also termed JL)×AVROS, (8) JL×DA, (9) JL×Highland Research Unit (also termed HRU), (10) JL×Institut pour Research sur les Huiles et Oléagineux (also termed IRHO), (11) (JL×HRU)×AVROS, (12) NIFOR×AVROS, (13) (NIFOR×DA)1, (14) (NIFOR×DA)2, (15) NIFOR×IRHO, (16) Nigerian×AVROS, (17) Serdang Avenue×AVROS, (18) Ulu Remis (also termed UR)×AVROS, (19) UR×DA, (20) UR×IRHO, (21) UR×Lobe, (22) (UR×NIFOR)1, (23) (UR×NIFOR)2, (24) (UR×NIFOR)3, (25) UR×Serdang AVROS, (26) UR×Serdang pisifera, and (27) BD×AVROS. Accordingly, in some examples the population of oil palm plants comprises: (1) (BD×NIFOR)×Jenderata, (2) Deli×AVROS, (3) Deli×Ekona, (4) (Elaeis guineensis×Elaeis oleifera) hybrid×AVROS, (5) Ekona×AVROS, (6) GM)×DA, (7) JL×AVROS, (8) JL×DA, (9) JL×HRU, (10) JL×IRHO, (11) (JL×HRU)×AVROS, (12) NIFOR×AVROS, (13) (NIFOR×DA)1, (14) (NIFOR×DA)2, (15) NIFOR×IRHO, (16) Nigerian×AVROS, (17) Serdang Avenue×AVROS, (18) UR×AVROS, (19) UR×DA, (20) UR×IRHO, (21) UR×Lobe, (22) (UR×NIFOR)1, (23) (UR×NIFOR)2, (24) (UR×NIFOR)3, (25) UR×Serdang AVROS, (26) UR×Serdang pisifera, or (27) BD×AVROS, or a combination thereof.

The sample of the test oil palm plant can comprise any organ, tissue, cell, or other part of the test oil palm plant that includes sufficient genomic DNA of the test oil palm plant to allow for determination of one or more SNP genotypes of the test oil palm plant, e.g. the first SNP genotype. For example, the sample can comprise a leaf tissue, among other organs, tissues, cells, or other parts. As one of ordinary skill will appreciate, determining, from a sample of a test oil palm plant, one or more SNP genotypes of the test oil palm plant, is necessarily transformative of the sample. The one or more SNP genotypes cannot be determined, for example, merely based on appearance of the sample. Rather, determination of the one or more SNP genotypes of the test oil palm plant requires separation of the sample from the test oil palm plant and/or separation of genomic DNA from the sample.

Determination of the at least first SNP genotype can be carried out by any suitable technique, including, for example, whole genome resequencing with SNP calling, hybridization-based methods, enzyme-based methods, or other post-amplification methods, among others.

The first SNP genotype corresponds to a first SNP marker. A SNP marker is a SNP that can be used in genetic mapping.

The first SNP marker is located in a first quantitative trait locus (also termed QTL) for a high-oil-production trait. A QTL is a locus extending along a portion of a chromosome that contributes in determining a phenotype of a continuous character, i.e. in this case, the high-oil-production trait.

The high-oil-production trait relates to a trait of production of palm oil by the test oil palm plant upon reaching a mature state, e.g. reaching production phase, and upon being cultivated under conditions suitable for production of palm oil in a high amount, e.g. commercial cultivation, in an amount that is higher than average, with respect to the population of oil palm plants from which the test oil palm plant is sampled, also upon reaching a mature state and upon being cultivated under conditions suitable for production of palm oil in a high amount.

Considering a test oil plant that is a tenera oil palm plant, the high-oil-production trait can correspond, for example, to production of palm oil at greater than 3.67 tonnes of palm oil per hectare per year, i.e. above recent average yields for typical oil palm plants used in commercial production, which also are tenera oil palm plants, as discussed above. The high-oil production trait also can correspond, for example, to production of palm oil at greater than 10 tonnes of palm oil per hectare per year, i.e. above recent average yields for current best-progeny oil palm plants used in commercial production. The high-oil production trait also can correspond, for example, to production of palm oil at greater than 4, 5, 6, 7, 8, or 9 tonnes of palm oil per hectare per year, i.e. above yields that are intermediate between the recent average yields noted above. Considering a test oil palm plant that is a dura oil palm plant or a pisifera oil palm plant, the high-oil production trait can correspond to production of palm oil in correspondingly lower amounts, consistent with lower average yields obtained for dura and pisifera oil palm plants relative to tenera oil palm plants.

The high-oil-production trait can comprise decreased shell-to-fruit (also termed S/F), increased mesocarp-to-fruit (also termed M/F), or a combination thereof, in tenera oil palm plants. Shell thickness and mesocarp thickness can be indicators of palm oil yield. More particularly, pre-planting selection of oil palm seed materials that will yield oil palm plants having fruits having a thinner shell and a thicker mesocarp is preferable for obtaining high-oil-yielding oil palm plants.

Regarding shell-to-fruit, as discussed above tenera fruits have thin shells, typically of 8% to 10% of the fruit by weight, i.e. S/F of 8% to 10% (shell weight/fruit weight). Moreover, it has been observed that palm oil yield tends to increase with decreasing shell-to-fruit for tenera oil palm plants. In addition, shell-to-fruit also generally is highly heritable. Shell thickness, measured as S/F (%), is inversely correlated to mesocarp thickness, measured as M/F (%). Thus, breeders are keen to select and produce tenera oil palm plants having fruits with thinner shells, so that the fruits have relatively more mesocarp for higher oil yield. Thus, a relatively low S/F is an indicator of relatively high production of palm oil for tenera oil palm plants.

Regarding mesocarp-to-fruit, as also discussed above, for tenera fruits the ratio of mesocarp to fruit typically is comparatively high, in the range of 60% to 80%, i.e. M/F of 60% to 80% (mesocarp weight/fruit weight). Moreover, it has been observed that palm oil yield tends to increase with increasing mesocarp-to-fruit for tenera oil palm plants. In addition, mesocarp-to-fruit also is highly heritable. Mesocarp-to-fruit contributes to determining palm oil yield in combination with other bunch traits according to the following formula: Yield of palm oil/year=(bunch number/year)×(average bunch weight)×(fruitlet/bunch)×(ratio of mesocarp to fruit)×(ratio of dry mesocarp to wet mesocarp)×(ratio of oil to dry mesocarp). Thus, breeders also are keen to select and produce tenera oil palm plants having fruits with thicker mesocarp, again so that the fruits have relatively more mesocarp for higher oil yield. Thus, a relatively high MN also is an indicator of relatively high production of palm oil for tenera oil palm plants.

Accordingly, in some examples the high-oil-production trait comprises decreased shell-to-fruit in tenera oil palm plants. Also, in some examples the high-oil-production trait comprises increased mesocarp-to-fruit in tenera oil palm plants. Also, in some examples the high-oil-production trait comprises decreased shell-to-fruit and increased mesocarp-to-fruit in tenera oil palm plants.

The first SNP marker is associated, after stratification and kinship correction, with the high-oil-production trait with a genome-wide −log₁₀(p-value) of at least 7.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 7.0 in the population.

A first SNP marker being associated, after stratification and kinship correction, with a trait with a genome-wide −log₁₀(p-value) of at least 7.0 in a population indicates that a high likelihood exists that the first SNP maker and the trait are associated.

A p-value is the probability of observing a test statistic, in this case relating to association of a SNP marker, e.g. the first SNP marker or the first other SNP marker, and the high-oil-production trait, equal to or greater than a test statistic actually observed, if the null hypothesis is true and thus there is no association, as discussed, for example, by Bush & Moore, Chapter 11: Genome-Wide Association Studies, PLOS Computational Biology 8(12):e1002822, 1-11 (2012). A genome-wide −log₁₀(p-value) corresponds to a p-value expressed on a logarithmic scale, for convenience, and corrected to take into account the effective number of statistical tests that have been carried out, based on multiple tests for association conducted with respect to an entire genome of a corresponding specific population, also as discussed by Bush & Moore (2012). Accordingly, a genome-wide −log₁₀(p-value) that is relatively high indicates that the likelihood that the observed test statistic, relating to association, would have been observed in the absence of association is extremely low.

Stratification and kinship correction are taken into account in determining the association. As noted above, stratification and kinship correction reduce false-positive signals due to recent common ancestry of small groups of individuals within the population of oil palm plants from which the test oil palm plant is sampled, thereby making practical the method for predicting palm oil yield of a test oil palm plant based on association.

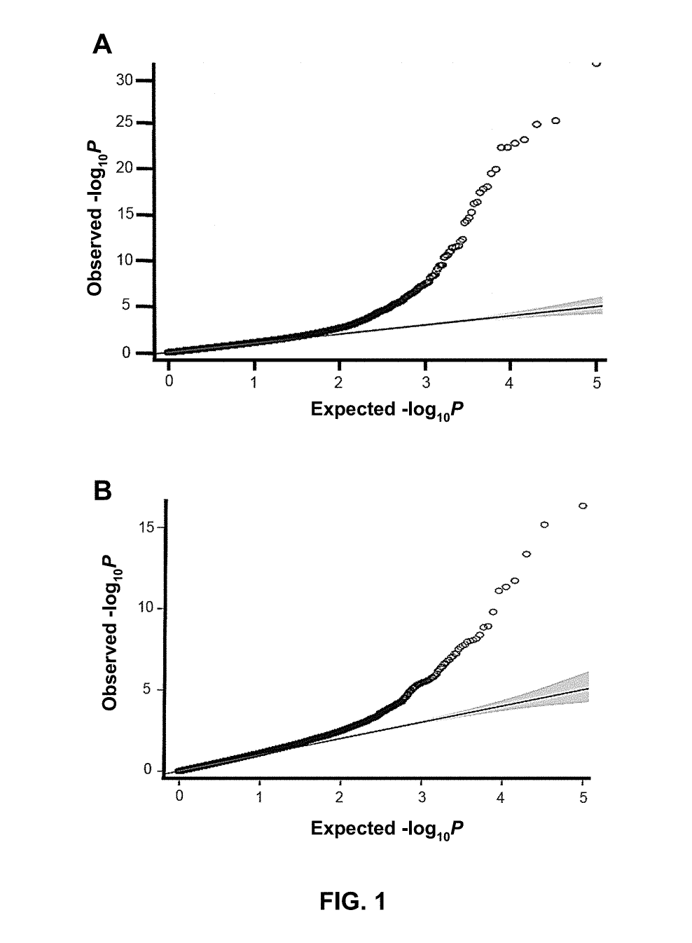

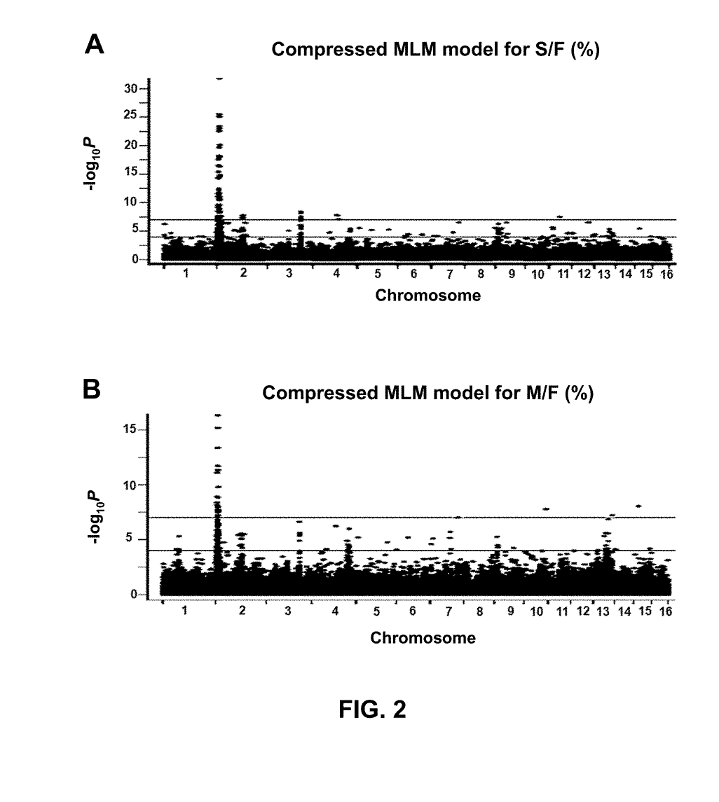

Considering the above-mentioned genome resequencing and genome-wide association studies of the 4,623 oil palm plants of the 27 oil palm origins in more detail, the genome-wide association study (also termed GWAS) was performed using a compressed mixed linear model (also termed MLM) with population parameters previously determined (P3D), to address the problem of genomic inflations using group kinship matrix. As shown in FIG. 1, the Q-Q plots in the 27 oil palm origins showed that deviation of the observed statistics from the null expectation were delayed significantly. As shown in FIG. 2, the chromosomal distribution of the resulting SNPs for the 27 oil palm origins can be visualized in Manhattan plots. Based on this approach, 68 SNPs that are informative with respect to S/F, M/F, or both were identified after excluding markers that overlapped in the 27 oil palm origins.

Stratification and kinship correction can be applied similarly regarding other oil palm populations too.

Accordingly, for example, the first SNP marker being located in a first QTL for a high-oil-production trait and being associated, after stratification and kinship correction, with the high-oil-production trait with a genome-wide −log₁₀(p-value) of at least 7.0 in the population can be a SNP marker for which association with the high-oil-production trait (i) has been confirmed based on a model that is not a naive model and/or (ii) would be confirmed based on a model that is not a naive model. Also for example, the first SNP marker being located in a first QTL for a high-oil-production trait and being associated, after stratification and kinship correction, with the high-oil-production trait with a genome-wide −log₁₀(p-value) of at least 7.0 in the population can be a SNP marker for which association with the high-oil-production trait (i) has been confirmed based on a compressed mixed linear model with population parameters previously determined, carried out using principal component analysis and a group kinship matrix and/or (ii) would be confirmed based on a compressed mixed linear model with population parameters previously determined, carried out using principal component analysis and a group kinship matrix.

A first SNP marker having a linkage disequilibrium r² value of at least 0.2 with respect to a first other SNP marker that is linked thereto and associated, after stratification and kinship correction, with the high-oil-production trait with a genome-wide −log₁₀(p-value) of at least 7.0 in the population indicates the following. First, a high likelihood exists that an allele of the first SNP marker and an allele of the first other SNP marker are in linkage disequilibrium. Second, a high likelihood exists that the first other SNP marker and the trait are associated. In this regard, a linkage disequilibrium r² value relates to measuring likelihood that two loci are in linkage disequilibrium as an average pairwise correlation coefficient.

Accordingly, in some examples the first SNP marker is associated, after stratification and kinship correction, with the high-oil-production trait with a genome-wide −log₁₀(p-value) of at least 7.0 in the population. Also, in some examples the first SNP marker has a linkage disequilibrium r² value of at least 0.2 with respect to a first other SNP marker that is linked thereto and associated, after stratification and kinship correction, with the high-oil-production trait with a genome-wide −log₁₀(p-value) of at least 7.0 in the population. Also, in some examples both apply.

The first QTL can be a region of the oil palm genome corresponding to one of:

(1) QTL region 1, extending from nucleotide 1516571 to 4215826 of chromosome 2;
(2) QTL region 2, extending from nucleotide 4858549 to 5594262 of chromosome 2;
(3) QTL region 3, extending from nucleotide 33949264 to 34110104 of chromosome 2;
(4) QTL region 4, extending from nucleotide 43405853 to 43834266 of chromosome 3;
(5) QTL region 5, extending from nucleotide 44126148 to 44193097 of chromosome 3;
(6) QTL region 6, extending from nucleotide 30702027 to 31148630 of chromosome 4;
(7) QTL region 7, extending from nucleotide 33166529 to 33451554 of chromosome 4;
(8) QTL region 8, extending from nucleotide 35906266 to 36257708 of chromosome 7;
(9) QTL region 9, extending from nucleotide 29233675 to 29612202 of chromosome 10;
(10) QTL region 10, extending from nucleotide 13470988 to 13734716 of chromosome 11;
(11) QTL region 11, extending from nucleotide 24620951 to 24989005 of chromosome 13; or
(12) QTL region 12, extending from nucleotide 6941783 to 7160542 of chromosome 15.

The numbering of chromosomes, also termed linkage groups, and nucleotides thereof is in accordance with a 1.8 gigabase genome sequence of the African oil palm E. guineensis as described by Singh et al., Nature 500335-339 (2013) and the supplementary information noted therein, indicating that the E. guineensis BioProject is available for download at http://genomsawit.mpob.gov.my and has been registered at the NCBI under BioProject accession PRJNA192219 and that the Whole Genome Shotgun project has been deposited at DDBJ/EMBL/GenBank under the accession ASJS00000000.

For reference, QTL region 1 corresponds to the region of chromosome 2 of the genome of oil palm extending from the 5′ end of SEQ ID NO: 1 to the 3′ end of SEQ ID NO: 2. Similarly, QTL region 2 corresponds to the region of chromosome 2 extending from the 5′ end of SEQ ID NO: 3 to the 3′ end of SEQ ID NO: 4. QTL region 3 corresponds to the region of chromosome 2 extending from the 5′ end of SEQ ID NO: 5 to the 3′ end of SEQ ID NO: 6. QTL region 4 corresponds to the region of chromosome 3 extending from the 5′ end of SEQ ID NO: 7 to the 3′ end of SEQ ID NO: 8. QTL region 5 corresponds to the region of chromosome 3 extending from the 5′ end of SEQ ID NO: 9 to the 3′ end of SEQ ID NO: 10. QTL region 6 corresponds to the region of chromosome 4 extending from the 5′ end of SEQ ID NO: 11 to the 3′ end of SEQ ID NO: 12. QTL region 7 corresponds to the region of chromosome 4 extending from the 5′ end of SEQ ID NO: 13 to the 3′ end of SEQ ID NO: 14. QTL region 8 corresponds to the region of chromosome 7 extending from the 5′ end of SEQ ID NO: 15 to the 3′ end of SEQ ID NO: 16. QTL region 9 corresponds to the region of chromosome 10 extending from the 5′ end of SEQ ID NO: 17 to the 3′ end of SEQ ID NO: 18. QTL region 10 corresponds to the region of chromosome 11 extending from the 5′ end of SEQ ID NO: 19 to the 3′ end of SEQ ID NO: 20. QTL region 11 corresponds to the region of chromosome 13 extending from the 5′ end of SEQ ID NO: 21 to the 3′ end of SEQ ID NO: 22. QTL region 12 corresponds to the region of chromosome 15 extending from the 5′ end of SEQ ID NO: 23 to the 3′ end of SEQ ID NO: 24.

The method also comprises a step of (ii) comparing the first SNP genotype of the test oil palm plant to a corresponding first reference SNP genotype indicative of the high-oil-production trait in the same genetic background as the population. The genetic background that is the same as the population can correspond, for example, to a population based on crossing oil palm plants of the same types as used to generate the population from which the test oil palm plant is sampled. Thus, for example, the genetic background that is the same as the population can correspond to one or more the 27 oil palm origins noted above, i.e. to (1) (BD×NIFOR)×Jenderata, (2) Deli×AVROS, (3) Deli×Ekona, (4) (Elaeis guineensis×Elaeis oleifera) hybrid×AVROS, (5) Ekona×AVROS, (6) GM)×DA, (7) JL×AVROS, (8) JL×DA, (9) JL×HRU, (10) JL×IRHO, (11) (JL×HRU)×AVROS, (12) NIFOR×AVROS, (13) (NIFOR×DA)1, (14) (NIFOR×DA)2, (15) NIFOR×IRHO, (16) Nigerian×AVROS, (17) Serdang Avenue×AVROS, (18) UR×AVROS, (19) UR×DA, (20) UR×IRHO, (21) UR×Lobe, (22) (UR×NIFOR)1, (23) (UR×NIFOR)2, (24) (UR×NIFOR)3, (25) UR×Serdang AVROS, (26) UR×Serdang pisifera, or (27) BD×AVROS, or a combination thereof. The genetic background that is the same as the population also can correspond, for example, to a population based on crossing the same individual oil palm plants used to generate the population from which the test oil palm plant is sampled. The genetic background that is the same as the population also can correspond, for example, to the same actual population from which the test oil palm plant is sampled.

The first reference SNP genotype indicative of the high-oil-production trait in the same genetic background as the population can correspond to the same SNP as the first SNP genotype, i.e. both can correspond to the same polymorphic variation with respect to a single nucleotide that occurs at a particular locus of a particular chromosome. The first reference SNP genotype can comprise one or more SNP alleles that, alone or together, indicate a higher likelihood that the test oil palm plant thereof exhibits, if mature, or will exhibit, upon reaching maturity, the high-oil-production trait, in comparison to oil palm plants of the same population that lack the one or more SNP alleles.

The method also comprises a step of (iii) predicting palm oil yield of the test oil palm plant based on the extent to which the first SNP genotype of the test oil palm plant matches the corresponding first reference SNP genotype. The first SNP genotype of the test oil palm plant can match the corresponding first reference SNP genotype based on both SNP genotypes sharing at least a first SNP allele indicative of the high-oil-production trait in the same genetic background as the population. In some examples the first SNP genotype and the first reference SNP genotype are heterozygous for the first allele indicative of the high-oil production trait, i.e. both have only one copy of the SNP allele. Also, in some examples the first SNP genotype and the first reference SNP genotype are homozygous for the first allele indicative of the high-oil production trait, i.e. both have two copies of the SNP allele. Also, in some examples the first SNP genotype is heterozygous for the first allele indicative of the high-oil production trait and the first reference SNP genotype is homozygous for the first allele indicative of the high-oil production trait. Also, in some examples the first SNP genotype is homozygous for the first allele indicative of the high-oil production trait and the first reference SNP genotype is heterozygous for the first allele indicative of the high-oil production trait.

The step of predicting palm oil yield of the test oil palm plant can further comprise applying a model, such as a genotype model, a dominant model, or a recessive model, among others, in order to facilitate the predicting. A genotype model tests the association of a trait, e.g. a high-oil production trait, with the presence of a SNP allele, either a major allele (A) or a minor allele (a). A dominant model tests the association of a trait, e.g. a high-oil production trait, with the presence of a SNP allele either as a homozygous genotype or a heterozygous genotype, e.g. the major allele either as a homozygous genotype (e.g. A/A) or a heterozygous genotype (e.g. A/a). A recessive model tests the association of a trait, e.g. a high-oil production trait, with the presence of a SNP allele as a homozygous genotype, e.g. the minor allele as a homozygous genotype (a/a). Accordingly, in some examples, the predicting of palm oil yield of the test oil palm plant further comprises applying a genotype model. Also in some examples, the predicting of palm oil yield of the test oil palm plant further comprises applying a dominant model. Also in some examples, the predicting of palm oil yield of the test oil palm plant further comprises applying a recessive model.

The degree to which a particular SNP genotype of a SNP marker in QTL regions 1 to 12 can be useful for predicting palm oil yield of a test oil palm plant can depend on the source and breeding history of the breeding materials used to generate the population from which the test oil palm is sampled, including for example the extent to which one or more high-yield variant alleles that result in increases in palm oil yield have arisen within QTL regions 1 to 12 of the breeding materials and/or sources thereof used to generate the population, as well as the proximity of the one or more high-yield variant alleles to SNPs and the extent to which recombination has occurred between the SNPs and the high-yield variant alleles since the high-yield variant alleles arose. Factors such as proximity between a high-yield variant allele that promotes a high-oil-production trait and a SNP allele, a low number of generations since the high-yield variant allele arose, and a strong positive effect of the high-yield variant allele on palm oil production can tend to increase the degree to which a particular SNP can be informative. These factors can vary, for example, depending on whether a high-yield variant allele is dominant or recessive, and thus whether a genotype model, a dominant model, or a recessive model may appropriately be applied with respect to a corresponding SNP allele. These factors also can vary, for example, between different populations generated by crosses of different individual palm plants.

The step of predicting palm oil yield of the test oil palm plant can be used advantageously not just to predict the palm oil yield of the test oil palm plant itself, but also to predict palm oil yields of progeny thereof. In this regard, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis.

The method for predicting palm oil yield of a test oil palm plant can be used by focusing on particular QTLs, or combinations thereof, with respect to test oil palm plants derived from particular breeding materials.

For example, in some examples the first QTL corresponds to one of QTL regions 1, 2, 3, 4, 5, 6, 7, or 10, the high-oil-production trait comprises decreased shell-to-fruit, and step (iii) further comprises applying a genotype model, thereby predicting the palm oil yield of the test oil palm plant.

Also, in some examples the first QTL corresponds to one of QTL regions 1, 8, 9, 11, or 12, the high-oil-production trait comprises increased mesocarp-to-fruit, and step (iii) further comprises applying a genotype model, thereby predicting the palm oil yield of the test oil palm plant.

Also in some examples the first QTL corresponds to QTL region 1, the high-oil-production trait comprises decreased shell-to-fruit and increased mesocarp-to-fruit, and step (iii) further comprises applying a genotype model, thereby predicting the palm oil yield of the test oil palm plant.

Also in some examples the first QTL corresponds to QTL region 1, the high-oil-production trait comprises decreased shell-to-fruit, and step (iii) further comprises applying a dominant model, thereby predicting the palm oil yield of the test oil palm plant.

Also in some examples the first QTL corresponds to QTL region 1, the high-oil-production trait comprises decreased shell-to-fruit, and step (iii) further comprises applying a recessive model, thereby predicting the palm oil yield of the test oil palm plant.

Also in some examples the first QTL corresponds to QTL region 1, the high-oil-production trait comprises increased mesocarp-to-fruit, and step (iii) further comprises applying a recessive model, thereby predicting the palm oil yield of the test oil palm plant.

Also in some examples the first QTL corresponds to QTL region 1, the high-oil-production trait comprises decreased shell-to-fruit and increased mesocarp-to-fruit, and step (iii) further comprises applying a recessive model, thereby predicting the palm oil yield of the test oil palm plant.

As noted above, crossing dura and pisifera gives rise to palms with a third fruit type, the tenera. As also noted, tenera are typically used as commercial planting materials. Accordingly, in some examples the test oil palm plant is a tenera candidate agricultural production plant. In some examples the population of oil palm plants comprises a commercially relevant Ulu Remis dura×AVROS pisifera population, and the test oil palm plant is a tenera candidate agricultural production plant. Also, in some examples the population of oil palm plants comprises a commercially relevant Banting dura×AVROS pisifera population, and the test oil palm plant is a tenera candidate agricultural production plant.

As also noted above, oil palm breeding is primarily aimed at selecting for improved parental dura and pisifera breeding stock palms for production of superior tenera commercial planting materials. As also noted, parental dura breeding populations are generated by crossing among selected dura palms, whereas pisifera palms are normally female sterile and thus breeding populations thereof must be generated by crossing among selected teneras or by crossing selected teneras with selected pisiferas. Accordingly, in some examples the test oil palm plant is a plant for mother palm selection and propagation, a plant for introgressed mother palm selection and propagation, or a plant for pollen donor selection and propagation. In some examples, the population of oil palm plants comprises an Ulu Remis dura×Ulu Remis dura population, and the test oil palm plant is a plant for mother palm selection and propagation. Also in some examples, the population of oil palm plants comprises Ulu Remis dura×Ulu Remis dura population, and the test oil palm plant is a plant for introgressed mother palm selection and propagation. Also in some examples, the population of oil palm plants comprises an Ulu Remis dura×Banting dura population, and the test oil palm plant is a plant for mother palm selection and propagation. Also in some examples, the population of oil palm plants comprises a Banting dura×Banting dura population, and the test oil palm plant is a plant for mother palm selection and propagation. Also in some examples, the population of oil palm plants comprises a Banting dura×Banting dura population, and the test oil palm plant is a plant for introgressed mother palm selection and propagation. Also in some examples, the population of oil palm plants comprises an AVROS pisifera×AVROS tenera population, and the test oil palm plant is a plant for pollen donor selection and propagation. Also in some examples, the population of oil palm plants comprises an AVROS tenera×AVROS tenera population, and the test oil palm plant is a plant for pollen donor selection and propagation.

The method for predicting palm oil yield of a test oil palm plant also can be carried out by determining additional SNP genotypes, comparing the additional SNP genotypes to corresponding reference genotypes indicative of the high-oil-production trait, and further predicting palm oil yield of the test oil palm plant based on the extent to which the additional SNP genotypes match the corresponding reference SNP genotypes. This is because each SNP genotype can reflect a high-yield variant allele that contributes to a high-oil-production trait additively and/or synergistically with respect to the others.

Accordingly, in some examples step (i) further comprises determining, from the sample of the test oil palm plant, at least a second SNP genotype of the test oil palm plant, the second SNP genotype corresponding to a second SNP marker, the second SNP marker (a) being located in a second QTL for the high-oil-production trait and (b) being associated, after stratification and kinship correction, with the high-oil-production trait with a genome-wide −log₁₀(p-value) of at least 7.0 in the population or having a linkage disequilibrium r² value of at least 0.2 with respect to a second other SNP marker that is linked thereto and associated, after stratification and kinship correction, with the high-oil-production trait with a genome-wide −log₁₀(p-value) of at least 7.0 in the population. Moreover, in these examples step (ii) further comprises comparing the second SNP genotype of the test oil palm plant to a corresponding second reference SNP genotype indicative of the high-oil-production trait in the same genetic background as the population. In addition, in these examples the second QTL corresponds to one of QTL regions 1 to 12, with the proviso that the first QTL and the second QTL correspond to different QTL regions. In some of these examples, step (iii) further comprises predicting palm oil yield of the test oil palm plant based on the extent to which the second SNP genotype of the test oil palm plant matches the corresponding second reference SNP genotype. Also, in some of these examples the high-oil-production trait can comprise decreased shell-to-fruit (also termed S/F), increased mesocarp-to-fruit (also termed M/F), or a combination thereof, in tenera oil palm plants, as discussed above.

Also in some examples, step (i) further comprises determining, from the sample of the test oil palm plant, at least a third SNP genotype to a twelfth SNP genotype of the test oil palm plant, the third SNP genotype to the twelfth SNP genotype corresponding to a third SNP marker to a twelfth SNP marker, respectively, the third SNP marker to the twelfth SNP marker (a) being located in a third QTL to a twelfth QTL, respectively, for the high-oil-production trait and (b) being associated, after stratification and kinship correction, with the high-oil-production trait with a genome-wide −log₁₀(p-value) of at least 7.0 in the population or having linkage disequilibrium r² values of at least 0.2 with respect to a third other SNP marker to a twelfth other SNP marker, respectively, that are linked thereto and associated, after stratification and kinship correction, with the high-oil-production trait with a genome-wide −log₁₀(p-value) of at least 7.0 in the population. Moreover, in these examples step (ii) further comprises comparing the third SNP genotype to the twelfth SNP genotype of the test oil palm plant to a corresponding third reference SNP genotype to a corresponding twelfth reference SNP genotype, respectively, indicative of the high-oil-production trait in the same genetic background as the population. In addition, in these examples the third QTL to the twelfth QTL each correspond to one of QTL regions 1 to 12, with the proviso that the first QTL to the twelfth QTL each correspond to different QTL regions. In some of these examples, step (iii) further comprises predicting palm oil yield of the test oil palm plant based on the extent to which the third SNP genotype to the twelfth SNP genotype of the test oil palm plant match the corresponding third reference SNP genotype to the corresponding twelfth reference SNP genotype, respectively. Also, in some of these examples the high-oil-production trait can comprise decreased shell-to-fruit (also termed S/F), increased mesocarp-to-fruit (also termed M/F), or a combination thereof, in tenera oil palm plants, as discussed above.

Also provided is a method of selecting a high-palm-oil-yielding oil palm plant for agricultural production of palm oil. The method comprises a step of (a) predicting palm oil yield of a test oil palm plant. This step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 12, as described above. The method also comprises a step of (b) field planting the test oil palm plant for agricultural production of palm oil if the palm oil yield of the test oil palm plant is predicted to be higher than average for the population based on step (a).

Also provided is a method of selecting a high-palm-oil-yielding oil palm plant for cultivation in cell culture. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 12, as described above. The method also comprises a step of (b) subjecting at least one cell of the test oil palm plant to cultivation in cell culture if the palm oil yield of the test oil palm plant is predicted to be higher than average for the population based on step (a).

Also provided is a method of selecting a parental oil palm plant for use in breeding to obtain agricultural production plants or improved parental oil palm plants. As noted above, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 12, as described above. The method also comprises a step of (b) selecting the test oil palm plant for use in breeding if the palm oil yield of tenera progeny of the test oil palm plant is predicted to be higher than average for the population based on step (a).

The following examples are for purposes of illustration and are not intended to limit the scope of the claims.

### EXAMPLES

### Sampling and DNA Preparation

The sampling was conducted on 4,623 oil palm plants representing genome-wide association study (also termed GWAS) mapping populations derived from 27 oil palm origins, including a commercially relevant Ulu Remis dura×AVROS pisifera population and a commercially relevant Banting dura×AVROS pisifera population, among others. The 27 oil palm origins include the following: (1) (BD×NIFOR)×Jenderata, (2) Deli×AVROS, (3) Deli×Ekona, (4) (Elaeis guineensis×Elaeis oleifera) hybrid×AVROS, (5) Ekona×AVROS, (6) GM)×DA, (7) JL×AVROS, (8) JL×DA, (9) JL×HRU, (10) JL×IRHO, (11) (JL×HRU)×AVROS, (12) NIFOR×AVROS, (13) (NIFOR×DA)1, (14) (NIFOR×DA)2, (15) NIFOR×IRHO, (16) Nigerian×AVROS, (17) Serdang Avenue×AVROS, (18) UR×AVROS, (19) UR×DA, (20) UR×IRHO, (21) UR×Lobe, (22) (UR×NIFOR)1, (23) (UR×NIFOR)2, (24) (UR×NIFOR)3, (25) UR×Serdang AVROS, (26) UR×Serdang pisifera, and (27) BD×AVROS. The sample selection was based on a good representation of shell-to-fruit (also termed S/F) (%) and mesocarp-to-fruit (also termed M/F) (%) variants and pedigree recorded by the corresponding breeders. Total genomic DNA was isolated from unopened spear leaves using the DNAeasy (R) Plant Mini Kit (Qiagen, Limburg, Netherlands).

### Whole-Genome Re-Sequencing

The samples were pooled based on an equal molar concentration of DNA from each sample to form the sequencing DNA pool. A library was prepared for re-sequencing using HiSeq 2000 (TM) sequencing systems (Illumina, San Diego, Calif.) to generate 100-bp pair-end reads to a 35× genome coverage, resulting in 1,015,758,056 raw reads. The pair-end reads were trimmed, filtered, and aligned to the published oil palm genome, as described by Singh et al., Nature 500:335-339 (2013), using BWA Mapper, as published by Li & Durbin, Bioinformatics 26:589-595 (2010), with default parameters. A total of approximately 6,846,197 putative SNPs were then called and filtered using SAMtools, as published by Li et al., Bioinformatics 25:2078-2079 (2009), with parameters of minimal mapping quality score of the SNP being 25, minimal depth 3×, and minimal SNP distance from a gap of 2 bp. Of the putative SNPs, 1,085,204 SNPs that were generated from Elaeis oleifera were removed. Also removed were 746,092 SNPs based on coverage (minimal 17 or maximal 53), genotype quality with minimal score of 8, and/or minor allele frequency (also termed MAF)<0.05. The other filtering steps were performed to remove 5,274,000 SNPs based on technical requirement of Illumina, including removal of pairs of SNPs with distance less than 60 bp and ambiguous nucleotides. This yielded 664,136 quality SNPs. According to linkage disequilibrium, r² cutoff being set at 0.3, a total of 200 K SNPs with an average density of one SNP per 11 Kb were submitted to Illumina for design score calculation using Illumina's Assay Design Tool for Infinium (Illumina).

### SNP Genotyping

An OP 100K Infinium array (Illumina) was used to assay the GWAS mapping populations (˜250 ng DNA/sample). The overnight amplified DNA samples were then fragmented by a controlled enzymatic process that did not require gel electrophoresis. The re-suspended DNA samples were hybridized to BeadChips (Illumina) after an overnight incubation in a corresponding capillary flow-through chamber. Allele specific hybridizations were fluorescently labeled and detected by a BeadArray Reader (Illumina). The raw reads were then analyzed using GenomeStudio Data Analysis software (Illumina) for automated genotyping calling and quality control. To generate the genotypic dataset for GWAS, only the SNPs that had minor allele frequency>0.01 and >90% of call rate were accepted. The missing genotype of those SNPs was subsequently imputed based on the mean of each marker, in accordance with Endelman, Plant Genome 4:250-255 (2011).

### Genetic Stratification and Population Analyses

The individuals in the study were first split into different populations based on their respective backgrounds, which addressed population structure effect. Within each population, kinship correction was carried out using relationship matrix between the individuals, which addressed cryptic relatedness.

### Phenotypic Data Compilation and GWAS

S/F corresponds to shell (also termed seed coat) per fruit, typically expressed on a weight/weight percentage (also termed %) basis.

M/F corresponds to mesocarp per fruit, also typically expressed on a weight/weight percentage (also termed %) basis.

Additional measurements include the following. Oil per palm (also termed O/P) is measured as fresh fruit bunch (also termed FFB)×oil per bunch (also termed O/B). FFB corresponds to total weight of bunches produced per palm per year. Measurement of FFB is typically conducted in the field during bunch harvesting. O/B corresponds to oil content per bunch.

Measurements of S/F, M/F, and O/B are carried out according to industry practice, as described by Blaak et al., “Methods of bunch analysis,” Breeding and Inheritance in the Oil Palm (Elaeis guineensis Jacq.) Part II, Vol. 4:146-155 (J. W. Afr. Ins. Oil Palm Res., 1963), with modifications as described by Rao et al., “A Critical Reexamination of the Method of Bunch Analysis in Oil Palm Breeding,” Palm Oil Research Institute Malaysia Occ Paper 9:1-28 (1983).

Association analyses were conducted on the 4,623 oil palm plants of the 27 oil palm origins based on a compressed mixed linear model with P3D analysis according to Zhang et al., Nature Genetics 42355-360 (2010), in the rrBLUP program, in accordance with Endelman (2011). The total number of common SNPs was 92,057 SNPs with minor allele frequency>0.01. Genetic sub-structure resulting from cryptic relatedness was accounted for by including kinship matrix, in accordance with VanRaden, Journal of Dairy Science 91:4414-4423 (2008), as a random effect in the compressed mixed linear model method. The whole-genome significance −log₁₀(p-value) cutoff was fixed at ≥7 for the 27 populations, due to the complex nature of the S/F trait and the M/F trait. The quantile-quantile (Q-Q) plots and Manhattan plots were then constructed using an R package qqman, in accordance with Turner, qqman: An R package for visualizing GWAS results using Q-Q and Manhattan plots, available at http://biorxiv.org/content/early/2014/05/14/005165 (last accessed Nov. 15, 2014). Inflated false-positive signals were also evaluated for both methods according to the genomic inflated factor (GIF) estimated in an R package GenABEL, in accordance with Aulchenko et al. (2007).

### SNP Effects and Statistical Analyses

The significant SNPs according to −log₁₀(p-value)≥7.0 were further analyzed for the genotype model-based SNP effects on S/F trait and M/F trait. The effects were determined by the differences of the mean trait values of genotypes that are responsible for high S/F and M/F respectively versus low S/F and M/F values. The same analytical method was expanded to determine S/F association and/or M/F association with the presence of one SNP allele, either a major allele (A) or a minor allele (a) through dominance model (A/A+A/a, a/a) and recessive model (A/A, A/a+a/a).

### Genomic Selection

For genomic selection, SNP markers were sorted based on their association score to the S/F trait and/or M/F trait. Unique SNP markers were selected to define a range. Analyses were carried out with respect to SNP markers sorted based on their association score to the S/F trait and/or M/F trait, from high association to low association. Analyses also were carried out with respect to SNP markers in linkage disequilibrium with SNP markers sorted based on their association score to the S/F trait and/or M/F trait, from high association to low association. For the case of linkage disequilibrium, graphs were generated based on one random SNP per region of linkage disequilibrium, with a total of 1,000 cycles each for cross validations.

### Results

Oil production phenotype data for the 4,623 oil palm plants representing the GWAS mapping populations derived from the 27 oil palm origins, expressed as S/F (%) and M/F (%), are provided in TABLE 1. As can be seen, the 4,623 oil palm plants exhibited a mean S/F (%) of 10.977%, and a mean M/F (%) of 79.799%.

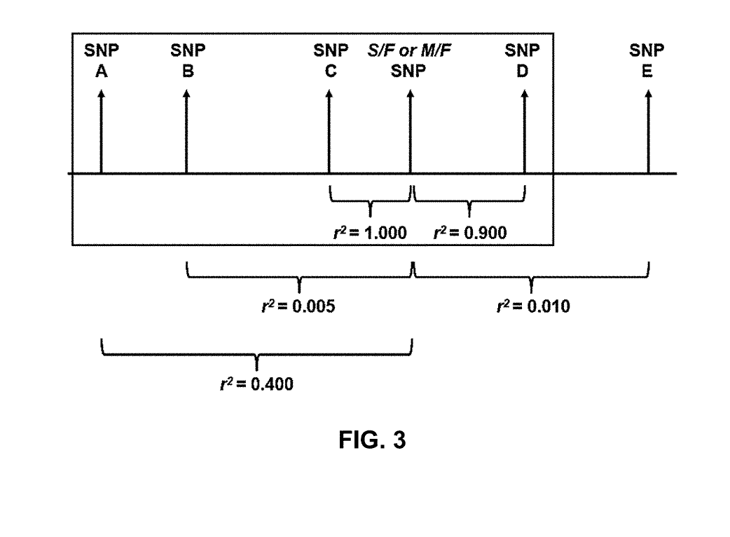

Twelve QTL regions for the S/F trait and the M/F trait in the 4,623 oil palm plants representing the GWAS mapping populations derived from the 27 oil palm origins were identified, as shown in TABLE 2, with elaboration in FIG. 3. The numbering of chromosomes and nucleotides thereof is in accordance with the 1.8 gigabase genome sequence of the African oil palm E. guineensis as described by Singh et al., Nature 500:335-339 (2013) and the supplementary information noted therein, as discussed above. The 12 QTL regions span 6,403,329 nucleotides, corresponding to approximately 0.36% of the oil palm genome.

Sixty-eight SNP markers that are informative with respect to S/F and/or M/F for the 4,623 oil palm plants representing the GWAS mapping populations derived from the 27 oil palm origins and that are located within the 12 QTLs were identified, as shown in TABLE 3, TABLE 4, TABLE 5, and TABLE 6. SNP identifying information and positional information is provided in TABLE 3. Major allele, minor allele, minor allele frequency, genotype of minimum shell-to-fruit (%), genotype of maximum shell-to-fruit (%), and genome-wide −log₁₀(p-value) for decreased shell-to-fruit (%) with respect to a genotype model, a dominant model, and a recessive model are provided in TABLE 4. Major allele, minor allele, minor allele frequency, genotype of minimum mesocarp-to-fruit (%), genotype of maximum mesocarp-to-fruit (%), and genome-wide −log₁₀(p-value) for decreased mesocarp-to-fruit (%) with respect to a genotype model, a dominant model, and a recessive model are provided in TABLE 5. Differences in mean shell-to-fruit (%) and mean mesocarp-to-fruit (%) for oil palm plants including a SNP allele associated with the high-oil-production trait versus oil palm plants lacking the SNP allele, with respect to the genotype model, are provided in TABLE 6.

As can be seen in TABLE 4 and TABLE 5, each of the SNP markers yielded a genome-wide −log₁₀(p-value) of at least 7.0 with respect to at least one of S/F or M/F with respect to at least one of a genotype model, a dominant model, or a recessive model. Indeed, many of the SNP markers yielded a genome-wide −log₁₀(p-value) of at least 7.0 in for both S/F and M/F and/or with respect to more than one of the models. Also, as can be seen in TABLE 6, for each of the SNP markers for which a minor SNP allele was detected, differences in mean percentage S/F and M/F for oil palm plants including a SNP allele associated with the high-oil-production trait (termed Min with respect to S/F and termed Max with respect to M/F) versus oil palm plants lacking the SNP allele (termed Max with respect to S/F and termed Min with respect to M/F), with respect to the genotype model in particular, ranged from 9.52% to 22.4% for S/F (%) and ranged from 68.20% to 82.70% for M/F (%). Various SNP markers are informative with respect to both S/F and M/F.

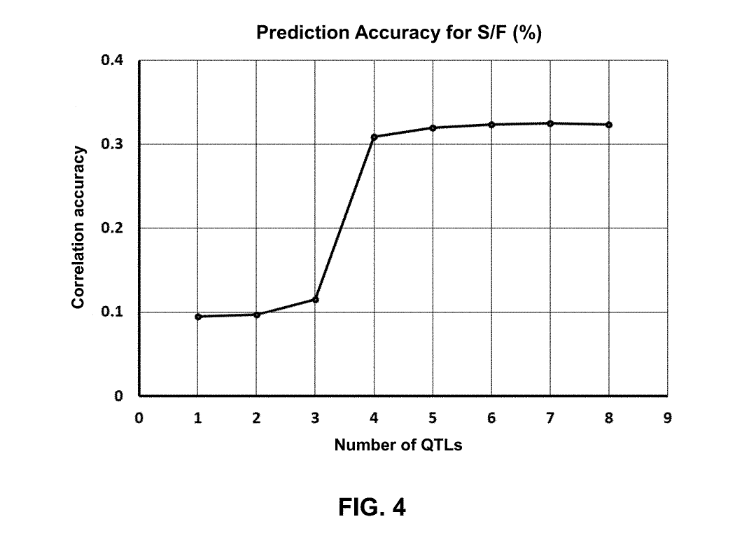

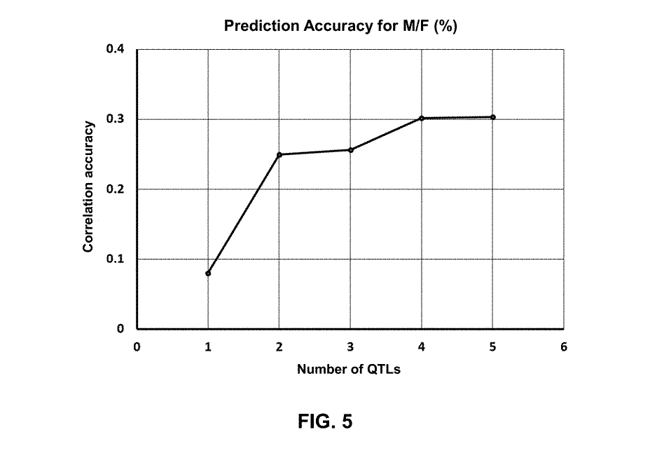

The 68 SNP markers can be used in various combinations to obtain increased prediction accuracy for both S/F and M/F. For example, as shown in TABLE 7 and FIG. 4, prediction accuracy for S/F (%) can be increased from 0.094660024%, as obtained based on use of one SNP marker, corresponding to SNP number 39 (SD_SNP_000035300) of QTL region 1, to 0.309159861%, as obtained based on use of four SNP markers, corresponding to SNP number 39 (SD_SNP_000035300) of QTL region 1, SNP number 59 (SD_SNP_000038060) of QTL region 4, SNP number 63 (SD_SNP_000033505) of QTL region 6, and SNP number 57 (SD_SNP_000042902) of QTL region 3. Also for example, as shown in TABLE 8 and FIG. 5, prediction accuracy for M/F (%) can be increased from 0.079364949%, as obtained based on use of one SNP marker, corresponding to SNP number 40 (SD_SNP_000015816) of QTL region 1, to 0.301288282%, as obtained based on use of four SNP markers, corresponding to SNP number 40 (SD_SNP_000015816) of QTL region 1, SNP number 68 (SD_SNP_000044156) of QTL region 12, SNP number 66 (SD_SNP_000006564) of QTL region 9, and SNP number 62 (SD_SNP_000010805) of QTL region 11. These results demonstrate an additive effect associated with use of the SNP markers in combination. Prediction accuracy can be improved further by using additional SNPs markers in combination.

TABLE 1
Shell-to-fruit (%) and mesocarp-to-fruit (%) of the
4,623 oil palm plants representing the GWAS mapping
populations derived from the 27 oil palm origins.

| | Mean | St. | Coef. | Median | Range |
|---|---|---|---|---|---|
| Trait | (%) | Dev. | Var. | (%) | (%) |
| Shell-to-fruit (%) | 10.977 | 2.766 | 0.252 | 10.800 | 18.500 |
| Mesocarp-to-fruit (%) | 79.799 | 4.638 | 0.058 | 80.100 | 30.600 |

TABLE 2
QTL regions 1 to 12: Chromosome and
nucleotide position information.

| | | Start | Stop | Length |
|---|---|---|---|---|
| QTL region | Chromosome | nucleotide | nucleotide | (Stop-Start + 1) |
| 1 | 2 | 1516571 | 4215826 | 2699256 |
| 2 | 2 | 4858549 | 5594262 | 735714 |
| 3 | 2 | 33949264 | 34110104 | 160841 |
| 4 | 3 | 43405853 | 43834266 | 428414 |
| 5 | 3 | 44126148 | 44193097 | 66950 |
| 6 | 4 | 30702027 | 31148630 | 446604 |
| 7 | 4 | 33166529 | 33451554 | 285026 |
| 8 | 7 | 35906266 | 36257708 | 351443 |
| 9 | 10 | 29233675 | 29612202 | 378528 |
| 10 | 11 | 13470988 | 13734716 | 263729 |
| 11 | 13 | 24620951 | 24989005 | 368055 |
| 12 | 15 | 6941783 | 7160542 | 218760 |

TABLE 3
SNP markers in QTL regions 1 to 12: SNP identifying
information and positional information.

| | | QTL | | |
|---|---|---|---|---|
| SNP No. | SNP ID | region | Chromosome | Position |
| 1 | SD_SNP_000009674 | 1 | 2 | 2839498 |
| 2 | SD_SNP_000034184 | 1 | 2 | 2663542 |
| 3 | SD_SNP_000034185 | 1 | 2 | 2665765 |
| 4 | SD_SNP_000001499 | 1 | 2 | 2382629 |
| 5 | SD_SNP_000035651 | 1 | 2 | 1759437 |
| 6 | SD_SNP_000035648 | 1 | 2 | 1771297 |
| 7 | SD_SNP_000022297 | 1 | 2 | 1846961 |
| 8 | SD_SNP_000012522 | 1 | 2 | 1885428 |
| 9 | SD_SNP_000031778 | 1 | 2 | 2126909 |
| 10 | SD_SNP_000032978 | 1 | 2 | 2221505 |
| 11 | SD_SNP_000032977 | 1 | 2 | 2222803 |
| 12 | SD_SNP_000052843 | 1 | 2 | 2248781 |
| 13 | SD_SNP_000022228 | 1 | 2 | 2252803 |
| 14 | SD_SNP_000049001 | 1 | 2 | 2278583 |
| 15 | SD_SNP_000012251 | 1 | 2 | 2334302 |
| 16 | SD_SNP_000039744 | 1 | 2 | 2420710 |
| 17 | SD_SNP_000051255 | 1 | 2 | 2441311 |
| 18 | SD_SNP_000034181 | 1 | 2 | 2667048 |
| 19 | SD_SNP_000042600 | 1 | 2 | 2695780 |
| 20 | SD_SNP_000042602 | 1 | 2 | 2703264 |
| 21 | SD_SNP_000018126 | 1 | 2 | 2716684 |
| 22 | SD_SNP_000014865 | 1 | 2 | 2743533 |
| 23 | SD_SNP_000013344 | 1 | 2 | 2823760 |
| 24 | SD_SNP_000009676 | 1 | 2 | 2832871 |
| 25 | SD_SNP_000052431 | 1 | 2 | 2916362 |
| 26 | SD_SNP_000052409 | 1 | 2 | 2930303 |
| 27 | SD_SNP_000045278 | 1 | 2 | 2950494 |
| 28 | SD_SNP_000053629 | 1 | 2 | 2981286 |
| 29 | SD_SNP_000053733 | 1 | 2 | 3012316 |
| 30 | SD_SNP_000050894 | 1 | 2 | 3058428 |
| 31 | SD_SNP_000044826 | 1 | 2 | 3088072 |
| 32 | SD_SNP_000044827 | 1 | 2 | 3091668 |
| 33 | SD_SNP_000054426 | 1 | 2 | 3097626 |
| 34 | SD_SNP_000049523 | 1 | 2 | 3102809 |
| 35 | SD_SNP_000049522 | 1 | 2 | 3106023 |
| 36 | SD_SNP_000032638 | 1 | 2 | 3138520 |
| 37 | SD_SNP_000032635 | 1 | 2 | 3164271 |
| 38 | SD_SNP_000032634 | 1 | 2 | 3178475 |
| 39 | SD_SNP_000035300 | 1 | 2 | 3208657 |
| 40 | SD_SNP_000015816 | 1 | 2 | 3231524 |
| 41 | SD_SNP_000015817 | 1 | 2 | 3234434 |
| 42 | SD_SNP_000015818 | 1 | 2 | 3244642 |
| 43 | SD_SNP_000020090 | 1 | 2 | 3261964 |
| 44 | SD_SNP_000042701 | 1 | 2 | 3516726 |
| 45 | SD_SNP_000042699 | 1 | 2 | 3525144 |
| 46 | SD_SNP_000021289 | 1 | 2 | 3628967 |
| 47 | SD_SNP_000021286 | 1 | 2 | 3642032 |
| 48 | SD_SNP_000025837 | 1 | 2 | 3758742 |
| 49 | SD_SNP_000025836 | 1 | 2 | 3768336 |
| 50 | SD_SNP_000025835 | 1 | 2 | 3772793 |
| 51 | SD_SNP_000044908 | 1 | 2 | 4074998 |
| 52 | SD_SNP_000044907 | 1 | 2 | 4077752 |
| 53 | SD_SNP_000020230 | 2 | 2 | 5097780 |
| 54 | SD_SNP_000025305 | 2 | 2 | 5363657 |
| 55 | SD_SNP_000042900 | 3 | 2 | 34075248 |
| 56 | SD_SNP_000042901 | 3 | 2 | 34075791 |
| 57 | SD_SNP_000042902 | 3 | 2 | 34078593 |
| 58 | SD_SNP_000038061 | 4 | 3 | 43585962 |
| 59 | SD_SNP_000038060 | 4 | 3 | 43590008 |
| 60 | SD_SNP_000003552 | 5 | 3 | 44177007 |
| 61 | SD_SNP_000045016 | 5 | 3 | 44193097 |
| 62 | SD_SNP_000010805 | 11 | 13 | 24755265 |
| 63 | SD_SNP_000033505 | 6 | 4 | 30941927 |
| 64 | SD_SNP_000030856 | 7 | 4 | 33272835 |
| 65 | SD_SNP_000032245 | 8 | 7 | 36083819 |
| 66 | SD_SNP_000006564 | 9 | 10 | 29394849 |
| 67 | SD_SNP_000032106 | 10 | 11 | 13717993 |
| 68 | SD_SNP_000044156 | 12 | 15 | 6941783 |

TABLE 4
SNP markers in QTL regions 1 to 12: Major allele, minor allele, minor allele
frequency, genotype of minimum shell-to-fruit (%), genotype of maximum shell-to-
fruit (%), and genome-wide −log₁₀(p-value) for decreased shell-to-fruit
(%) with respect to a genotype model, a dominant model, and a recessive
model. SNP numbering is in accordance with Table 3.

| | | | Minor | Min. | Max. | [−log₁₀(p-value)] for | | |
|---|---|---|---|---|---|---|---|---|
| SNP | Major | Minor | allele | S/F | S/F | decreased shell-to-fruit (%) | | |
| No. | allele | allele | freq. | geno. | geno. | Genotype | Dominant | Recessive |
| 1 | C | A | 0.289 | A/C | C/C | 9.552 | 0.000 | 8.421 |
| 2 | C | A | 0.2303 | A/C | A/A | 10.505 | 0.000 | 9.411 |
| 3 | A | C | 0.4547 | C/C | A/A | 11.490 | 8.208 | 7.565 |
| 4 | G | A | 0.1151 | G/G | A/A | 17.581 | 0.000 | 12.028 |
| 5 | G | A | 0.1283 | G/G | A/A | 7.368 | 0.000 | 5.906 |
| 6 | G | A | 0.1212 | G/G | A/G | 7.646 | 0.000 | 5.103 |
| 7 | G | A | 0.1783 | G/G | A/A | 7.004 | 0.000 | 2.271 |
| 8 | A | G | 0.2036 | A/A | G/G | 7.268 | 0.000 | 5.646 |
| 9 | A | C | 0.4742 | A/A | C/C | 9.589 | 4.995 | 8.071 |
| 10 | C | A | 0.3228 | A/A | C/C | 8.543 | 3.768 | 6.628 |
| 11 | A | G | 0.291 | A/A | G/G | 14.269 | 4.103 | 9.206 |
| 12 | A | G | 0.2108 | A/A | G/G | 11.103 | 0.000 | 11.508 |
| 13 | G | A | 0.4926 | G/G | A/A | 11.532 | 11.800 | 4.821 |
| 14 | A | G | 0.3232 | A/A | G/G | 9.188 | 1.927 | 7.977 |
| 15 | G | A | 0.1473 | G/G | A/A | 9.266 | 0.000 | 8.756 |
| 16 | A | G | 0.1336 | A/A | G/G | 15.395 | 0.000 | 12.353 |
| 17 | A | G | 0.1394 | A/A | G/G | 16.511 | 0.000 | 12.619 |
| 18 | A | G | 0.3148 | G/A | A/A | 11.722 | 0.000 | 8.301 |
| 19 | A | G | 0.3497 | G/A | G/G | 7.352 | 0.000 | 8.615 |
| 20 | G | A | 0.3487 | A/G | A/A | 8.390 | 0.000 | 10.433 |
| 21 | A | G | 0.2639 | G/A | A/A | 12.417 | 0.000 | 13.202 |
| 22 | A | C | 0.2534 | C/A | A/A | 12.202 | 0.000 | 11.570 |
| 23 | A | G | 0.409 | A/A | G/G | 8.394 | 8.159 | 3.005 |
| 24 | A | G | 0.3011 | G/A | A/A | 7.305 | 1.200 | 10.625 |
| 25 | C | A | 0.2237 | A/C | C/C | 9.596 | 0.000 | 8.138 |
| 26 | G | A | 0.2601 | A/G | G/G | 11.635 | 0.000 | 10.126 |
| 27 | C | A | 0.2649 | C/C | A/A | 9.605 | 0.000 | 10.837 |
| 28 | G | A | 0.3846 | A/G | A/A | 25.486 | 0.000 | 22.077 |
| 29 | G | A | 0.2129 | A/G | A/A | 11.613 | 0.000 | 11.878 |
| 30 | C | A | 0.4227 | A/C | C/C | 6.054 | 2.713 | 17.188 |
| 31 | G | A | 0.2129 | A/G | A/A | 17.961 | 0.000 | 12.531 |
| 32 | G | A | 0.4929 | G/G | A/A | 19.665 | 5.049 | 13.979 |
| 33 | A | G | 0.4796 | A/A | G/G | 23.007 | 5.167 | 18.883 |
| 34 | A | G | 0.2678 | A/A | G/G | 14.486 | 0.000 | 14.055 |
| 35 | A | C | 0.2648 | A/A | C/C | 10.686 | 0.000 | 10.250 |
| 36 | A | G | 0.1138 | A/A | G/G | 22.568 | 0.000 | 16.519 |
| 37 | C | A | 0.1627 | C/C | A/C | 22.541 | 0.000 | 22.336 |
| 38 | A | G | 0.05755 | G/A | G/G | 7.034 | 0.000 | 7.381 |
| 39 | A | G | 0.2953 | A/A | G/G | 31.785 | 5.232 | 22.543 |
| 40 | A | C | 0.2532 | C/A | C/C | 23.397 | 0.000 | 24.469 |
| 41 | A | G | 0.2234 | G/A | G/G | 8.239 | 0.000 | 8.508 |
| 42 | A | G | 0.2442 | G/G | A/A | 8.924 | 0.000 | 4.156 |
| 43 | A | C | 0.4702 | C/C | A/A | 18.199 | 13.996 | 8.498 |
| 44 | G | A | 0.2543 | A/G | G/G | 11.044 | 0.000 | 10.142 |
| 45 | C | A | 0.3308 | C/C | A/A | 16.351 | 5.002 | 8.967 |
| 46 | A | G | 0.3413 | G/A | A/A | 7.310 | 5.868 | 2.711 |
| 47 | G | A | 0.3505 | A/G | G/G | 8.461 | 4.970 | 2.748 |
| 48 | G | A | 0.1785 | A/G | G/G | 10.440 | 0.000 | 7.475 |
| 49 | A | C | 0.1966 | C/A | A/A | 10.668 | 0.000 | 8.079 |
| 50 | G | A | 0.2799 | G/G | A/A | 25.085 | 1.979 | 20.074 |
| 51 | A | G | 0.3255 | A/A | G/G | 20.122 | 8.825 | 8.797 |
| 52 | C | A | 0.381 | C/C | A/A | 14.791 | 4.912 | 6.827 |
| 53 | A | G | 0.2368 | A/A | G/G | 7.647 | 0.000 | 6.088 |
| 54 | G | A | 0.1073 | G/G | A/A | 7.639 | 0.000 | 3.049 |
| 55 | A | C | 0.3162 | A/A | C/C | 7.419 | 3.187 | 3.383 |
| 56 | A | G | 0.3166 | A/A | G/G | 7.321 | 3.251 | 3.612 |
| 57 | G | A | 0.3151 | G/G | A/A | 7.766 | 2.859 | 3.308 |
| 58 | A | G | 0.1425 | G/G | G/A | 8.146 | 0.000 | 3.435 |
| 59 | A | G | 0.1326 | G/G | G/A | 8.414 | 0.000 | 6.963 |
| 60 | G | A | 0.3228 | A/G | A/A | 7.146 | 4.094 | 1.324 |
| 61 | A | G | 0.4 | G/A | G/G | 7.510 | 5.273 | 1.005 |
| 62 | G | A | 0.2863 | A/G | G/G | 4.189 | 0.272 | 0.823 |
| 63 | G | A | 0.1906 | A/G | G/G | 7.787 | 0.000 | 0.032 |
| 64 | A | G | 0.1502 | A/A | G/G | 7.070 | 0.000 | 0.847 |
| 65 | G | A | 0.2913 | A/A | G/G | 6.510 | 0.500 | 1.428 |
| 66 | G | A | 0.08552 | A/A | A/G | 2.459 | 0.000 | 0.473 |
| 67 | C | A | 0.2453 | C/C | A/C | 7.507 | 0.000 | 0.600 |
| 68 | A | G | 0.2879 | G/A | G/G | 5.448 | 0.220 | 0.617 |

TABLE 5
SNP markers in QTL regions 1 to 12: Major allele, minor allele, minor allele
frequency, genotype of minimum mesocarp-to-fruit (%), genotype of maximum
mesocarp-to-fruit (%), and genome-wide −log₁₀(p-value) for increased mesocarp-to-fruit
(%) with respect to a genotype model, a dominant model, and a recessive model.
SNP numbering is in accordance with Table 3.

| | | | Minor | Min. | Max. | [−log₁₀(p-value)] for increased | | |
|---|---|---|---|---|---|---|---|---|
| SNP | Major | Minor | allele | M/F | M/F | mesocarp-to-fruit (%) | | |
| No. | allele | allele | freq. | geno. | geno. | Genotype | Dominant | Recessive |
| 1 | C | A | 0.289 | C/C | A/A | 8.020 | 0.000 | 6.546 |
| 2 | C | A | 0.2303 | C/C | A/C | 5.764 | 0.000 | 4.622 |
| 3 | A | C | 0.4547 | A/A | C/C | 4.939 | 3.469 | 4.047 |
| 4 | G | A | 0.1151 | A/A | G/G | 11.099 | 0.000 | 7.057 |
| 5 | G | A | 0.1283 | A/A | G/G | 4.378 | 0.000 | 3.348 |
| 6 | G | A | 0.1212 | A/G | A/A | 5.334 | 0.000 | 3.949 |
| 7 | G | A | 0.1783 | A/A | G/G | 4.252 | 0.000 | 1.454 |
| 8 | A | G | 0.2036 | G/G | A/A | 2.713 | 0.000 | 2.126 |
| 9 | A | C | 0.4742 | C/A | A/A | 8.383 | 2.547 | 6.145 |
| 10 | C | A | 0.3228 | C/C | A/A | 4.074 | 2.269 | 2.927 |
| 11 | A | G | 0.291 | G/G | A/A | 5.877 | 1.190 | 4.629 |
| 12 | A | G | 0.2108 | G/G | A/A | 3.770 | 0.000 | 4.123 |
| 13 | G | A | 0.4926 | A/A | G/G | 4.272 | 5.548 | 1.588 |
| 14 | A | G | 0.3232 | G/G | A/A | 4.977 | 0.831 | 4.737 |
| 15 | G | A | 0.1473 | A/A | G/G | 4.980 | 0.000 | 5.279 |
| 16 | A | G | 0.1336 | G/G | A/A | 8.142 | 0.000 | 6.392 |
| 17 | A | G | 0.1394 | G/G | A/A | 8.921 | 0.000 | 6.741 |
| 18 | A | G | 0.3148 | A/A | G/A | 5.473 | 0.000 | 3.890 |
| 19 | A | G | 0.3497 | G/G | G/A | 5.787 | 0.000 | 6.195 |
| 20 | G | A | 0.3487 | A/A | A/G | 6.603 | 0.000 | 7.345 |
| 21 | A | G | 0.2639 | A/A | G/A | 6.822 | 0.000 | 7.515 |
| 22 | A | C | 0.2534 | A/A | C/A | 6.144 | 0.000 | 6.309 |
| 23 | A | G | 0.409 | G/G | A/A | 4.515 | 5.644 | 0.990 |
| 24 | A | G | 0.3011 | A/A | G/G | 6.725 | 1.779 | 7.273 |
| 25 | C | A | 0.2237 | C/C | A/C | 5.429 | 0.000 | 5.360 |
| 26 | G | A | 0.2601 | G/G | A/G | 7.053 | 0.000 | 6.606 |
| 27 | C | A | 0.2649 | A/A | C/C | 4.291 | 0.000 | 5.031 |
| 28 | G | A | 0.3846 | G/G | A/G | 16.331 | 0.000 | 13.937 |
| 29 | G | A | 0.2129 | G/G | A/G | 7.495 | 0.000 | 7.071 |
| 30 | C | A | 0.4227 | C/C | A/C | 8.847 | 0.198 | 11.846 |
| 31 | G | A | 0.2129 | A/A | A/G | 11.345 | 0.000 | 7.230 |
| 32 | G | A | 0.4929 | A/G | G/G | 5.638 | 0.118 | 6.461 |
| 33 | A | G | 0.4796 | G/A | A/A | 6.405 | 0.108 | 7.866 |
| 34 | A | G | 0.2678 | G/G | A/A | 7.230 | 0.000 | 6.516 |
| 35 | A | C | 0.2648 | C/C | A/A | 5.321 | 0.000 | 5.119 |
| 36 | A | G | 0.1138 | G/G | A/A | 13.365 | 0.000 | 9.047 |
| 37 | C | A | 0.1627 | A/C | A/A | 7.962 | 0.000 | 7.483 |
| 38 | A | G | 0.05755 | G/G | G/A | 4.787 | 0.000 | 5.364 |
| 39 | A | G | 0.2953 | G/A | A/A | 11.720 | 0.877 | 10.213 |
| 40 | A | C | 0.2532 | C/C | C/A | 15.178 | 0.000 | 15.856 |
| 41 | A | G | 0.2234 | A/A | G/A | 5.909 | 0.000 | 6.123 |
| 42 | A | G | 0.2442 | A/A | G/G | 4.021 | 0.000 | 1.643 |
| 43 | A | C | 0.4702 | A/A | C/C | 7.724 | 6.683 | 3.578 |
| 44 | G | A | 0.2543 | G/G | A/G | 3.554 | 0.000 | 5.222 |
| 45 | C | A | 0.3308 | A/A | C/C | 5.515 | 1.059 | 4.015 |
| 46 | A | G | 0.3413 | A/A | G/A | 3.150 | 2.503 | 1.306 |
| 47 | G | A | 0.3505 | G/G | A/G | 3.349 | 2.132 | 1.301 |
| 48 | G | A | 0.1785 | G/G | A/A | 5.135 | 0.000 | 3.866 |
| 49 | A | C | 0.1966 | A/A | C/A | 6.420 | 0.000 | 4.031 |
| 50 | G | A | 0.2799 | A/G | G/G | 9.796 | 0.379 | 8.126 |
| 51 | A | G | 0.3255 | G/G | A/A | 7.623 | 3.327 | 3.145 |
| 52 | C | A | 0.381 | A/A | C/C | 6.292 | 1.953 | 2.873 |
| 53 | A | G | 0.2368 | G/G | A/A | 3.428 | 0.000 | 2.867 |
| 54 | G | A | 0.1073 | A/A | G/G | 4.205 | 0.000 | 2.032 |
| 55 | A | C | 0.3162 | C/C | A/A | 5.096 | 2.622 | 1.767 |
| 56 | A | G | 0.3166 | G/G | A/A | 5.077 | 2.770 | 1.916 |
| 57 | G | A | 0.3151 | A/A | G/G | 5.388 | 2.406 | 1.747 |
| 58 | A | G | 0.1425 | G/A | G/G | 5.353 | 0.000 | 2.579 |
| 59 | A | G | 0.1326 | G/A | G/G | 6.624 | 0.000 | 5.847 |
| 60 | G | A | 0.3228 | A/A | A/G | 5.610 | 2.081 | 1.985 |
| 61 | A | G | 0.4 | G/G | G/A | 5.503 | 2.943 | 1.414 |
| 62 | G | A | 0.2863 | G/G | A/A | 7.229 | 0.942 | 1.251 |
| 63 | G | A | 0.1906 | G/G | A/G | 6.233 | 0.000 | 0.382 |
| 64 | A | G | 0.1502 | G/G | G/A | 2.763 | 0.000 | 0.439 |
| 65 | G | A | 0.2913 | G/G | A/A | 7.008 | 0.494 | 1.650 |
| 66 | G | A | 0.08552 | G/G | A/A | 7.781 | 0.000 | 0.894 |
| 67 | C | A | 0.2453 | A/C | A/A | 2.232 | 0.000 | 0.046 |
| 68 | A | G | 0.2879 | G/G | A/A | 8.052 | 0.326 | 1.070 |

TABLE 6
SNP markers in QTL regions 1 to 12: Differences (termed δ) in mean
shell-to-fruit (%) and mean mesocarp-to-fruit (%) for oil palm plants
including a SNP allele associated with the high-oil-production trait
(termed Max) versus oil palm plants lacking the SNP allele (termed
Min), with respect to the genotype model. SNP numbering is in
accordance with Table 3.

| | SNP effects | | | SNP effects | | |
|---|---|---|---|---|---|---|
| | (Genotype Model): | | | (Genotype Model): | | |
| | Mean shell-to-fruit (%) | | | Mean mesocarp-to-fruit (%) | | |
| SNP No. | Min | Max | δ | Min | Max | δ |
| 1 | 10.350 | 11.620 | 1.270 | 78.840 | 80.780 | 1.940 |
| 2 | 10.190 | 12.280 | 2.090 | 79.010 | 80.750 | 1.740 |
| 3 | 10.030 | 11.960 | 1.930 | 78.560 | 80.860 | 2.300 |
| 4 | 10.570 | 14.870 | 4.300 | 74.970 | 80.470 | 5.500 |
| 5 | 10.640 | 14.210 | 3.570 | 76.240 | 80.380 | 4.140 |
| 6 | 10.620 | 12.090 | 1.470 | 77.890 | 82.700 | 4.810 |
| 7 | 10.640 | 13.780 | 3.140 | 75.510 | 80.390 | 4.880 |
| 8 | 10.380 | 13.220 | 2.840 | 78.540 | 80.480 | 1.940 |
| 9 | 10.470 | 11.310 | 0.840 | 79.590 | 80.350 | 0.760 |
| 10 | 10.410 | 11.600 | 1.190 | 78.900 | 81.310 | 2.410 |
| 11 | 10.050 | 12.750 | 2.700 | 78.190 | 80.830 | 2.640 |
| 12 | 10.500 | 13.870 | 3.370 | 77.310 | 80.540 | 3.230 |
| 13 | 9.880 | 12.130 | 2.250 | 78.130 | 81.320 | 3.190 |
| 14 | 9.930 | 12.960 | 3.030 | 77.100 | 81.200 | 4.100 |
| 15 | 10.590 | 13.260 | 2.670 | 76.390 | 80.490 | 4.100 |
| 16 | 10.570 | 14.600 | 4.030 | 75.900 | 80.470 | 4.570 |
| 17 | 10.520 | 14.600 | 4.080 | 75.900 | 80.520 | 4.620 |
| 18 | 10.500 | 11.550 | 1.050 | 79.190 | 80.320 | 1.130 |
| 19 | 10.590 | 13.270 | 2.680 | 77.930 | 80.520 | 2.590 |
| 20 | 10.590 | 13.210 | 2.620 | 78.030 | 80.520 | 2.490 |
| 21 | 10.280 | 11.650 | 1.370 | 78.990 | 80.640 | 1.650 |
| 22 | 10.250 | 11.650 | 1.400 | 78.910 | 80.740 | 1.830 |
| 23 | 9.980 | 12.080 | 2.100 | 77.570 | 81.220 | 3.650 |
| 24 | 10.190 | 11.730 | 1.540 | 78.660 | 80.740 | 2.080 |
| 25 | 10.270 | 11.510 | 1.240 | 79.070 | 80.760 | 1.690 |
| 26 | 10.250 | 11.680 | 1.430 | 78.890 | 80.730 | 1.840 |
| 27 | 10.080 | 11.950 | 1.870 | 77.980 | 80.790 | 2.810 |
| 28 | 10.580 | 12.680 | 2.100 | 77.700 | 80.450 | 2.750 |
| 29 | 10.040 | 11.890 | 1.850 | 78.940 | 80.990 | 2.050 |
| 30 | 10.470 | 12.280 | 1.810 | 77.510 | 80.440 | 2.930 |
| 31 | 10.000 | 12.030 | 2.030 | 78.570 | 81.050 | 2.480 |
| 32 | 9.530 | 12.330 | 2.800 | 79.620 | 81.040 | 1.420 |
| 33 | 9.570 | 12.320 | 2.750 | 79.590 | 80.950 | 1.360 |
| 34 | 9.950 | 14.400 | 4.450 | 75.920 | 81.090 | 5.170 |
| 35 | 10.020 | 12.880 | 2.860 | 78.220 | 80.970 | 2.750 |
| 36 | 10.570 | 22.400 | 11.830 | 68.200 | 80.460 | 12.260 |
| 37 | 10.480 | 12.060 | 1.580 | 78.400 | 80.690 | 2.290 |
| 38 | 10.700 | 11.620 | 0.920 | 78.620 | 79.920 | 1.300 |
| 39 | 9.900 | 12.650 | 2.750 | 78.670 | 80.940 | 2.270 |
| 40 | 10.030 | 12.370 | 2.340 | 78.220 | 80.980 | 2.760 |
| 41 | 10.030 | 12.050 | 2.020 | 78.820 | 81.040 | 2.220 |
| 42 | 9.520 | 11.570 | 2.050 | 79.060 | 81.310 | 2.250 |
| 43 | 9.530 | 12.240 | 2.710 | 78.210 | 81.630 | 3.420 |
| 44 | 10.190 | 11.830 | 1.640 | 78.740 | 80.790 | 2.050 |
| 45 | 9.910 | 12.720 | 2.810 | 78.780 | 80.930 | 2.150 |
| 46 | 10.620 | 11.480 | 0.860 | 79.350 | 80.210 | 0.860 |
| 47 | 10.630 | 11.440 | 0.810 | 79.360 | 80.190 | 0.830 |
| 48 | 10.030 | 11.500 | 1.470 | 79.100 | 82.700 | 3.600 |
| 49 | 10.110 | 11.440 | 1.330 | 79.150 | 80.930 | 1.780 |
| 50 | 9.940 | 12.630 | 2.690 | 78.650 | 80.860 | 2.210 |
| 51 | 9.920 | 13.010 | 3.090 | 78.090 | 80.920 | 2.830 |
| 52 | 9.740 | 12.890 | 3.150 | 77.820 | 81.270 | 3.450 |
| 53 | 10.300 | 14.080 | 3.780 | 77.390 | 80.430 | 3.040 |
| 54 | 10.680 | 14.300 | 3.620 | 76.950 | 80.240 | 3.290 |
| 55 | 10.780 | 11.520 | 0.740 | 78.550 | 80.100 | 1.550 |
| 56 | 10.780 | 11.520 | 0.740 | 78.540 | 80.110 | 1.570 |
| 57 | 10.780 | 11.510 | 0.730 | 78.560 | 80.110 | 1.550 |
| 58 | 10.020 | 11.640 | 1.620 | 78.840 | 81.050 | 2.210 |
| 59 | 10.020 | 11.770 | 1.750 | 78.570 | 81.050 | 2.480 |
| 60 | 10.660 | 11.980 | 1.320 | 78.850 | 80.350 | 1.500 |
| 61 | 10.440 | 11.940 | 1.500 | 78.670 | 80.630 | 1.960 |
| 62 | 10.970 | 11.110 | 0.140 | 79.340 | 80.620 | 1.280 |
| 63 | 10.870 | 10.890 | 0.020 | 79.830 | 80.040 | 0.210 |
| 64 | 10.900 | 13.900 | 3.000 | 76.620 | 80.000 | 3.380 |
| 65 | 10.460 | 11.380 | 0.920 | 79.400 | 81.150 | 1.750 |
| 66 | 10.490 | 11.270 | 0.780 | 79.560 | 82.310 | 2.750 |
| 67 | 10.700 | 11.130 | 0.430 | 79.800 | 82.700 | 2.900 |
| 68 | 10.470 | 11.830 | 1.360 | 77.890 | 80.440 | 2.550 |

TABLE 7
Additive effect on correlation accuracy for S/F (%) of using
from one SNP marker to eight SNP markers, corresponding to
the SNP Nos., SNP IDs, and QTL regions as indicated.

| Number | | | | |
|---|---|---|---|---|
| of QTLs | | | | Correlation |
| represented | | | | accuracy |
| in analysis | SNP | | QTL | for S/F |
| (additive) | No. | SNP ID | region | (%) |
| 1 | 39 | SD_SNP_000035300 | 1 | 0.095 |
| 2 | 59 | SD_SNP_000038060 | 4 | 0.097 |
| 3 | 63 | SD_SNP_000033505 | 6 | 0.115 |
| 4 | 57 | SD_SNP_000042902 | 3 | 0.309 |
| 5 | 53 | SD_SNP_000020230 | 2 | 0.319 |
| 6 | 61 | SD_SNP_000045016 | 5 | 0.324 |
| 7 | 67 | SD_SNP_000032106 | 10 | 0.325 |
| 8 | 64 | SD_SNP_000030856 | 7 | 0.324 |

TABLE 8
Additive effect on correlation accuracy for M/F (%) of using
from one SNP marker to five SNP markers, corresponding to
the SNP Nos., SNP IDs, and QTL regions as indicated.

| Number | | | | |
|---|---|---|---|---|
| of QTLs | | | | Correlation |
| represented | | | | accuracy |
| in analysis | SNP | | QTL | for M/F |
| (additive) | No. | SNP ID | region | (%) |
| 1 | 40 | SD_SNP_000015816 | 1 | 0.079 |
| 2 | 68 | SD_SNP_000044156 | 12 | 0.250 |
| 3 | 66 | SD_SNP_000006564 | 9 | 0.257 |
| 4 | 62 | SD_SNP_000010805 | 11 | 0.301 |
| 5 | 65 | SD_SNP_000032245 | 8 | 0.303 |

### INDUSTRIAL APPLICABILITY

The methods disclosed herein are useful for predicting oil yield of a test oil palm plant, and thus for improving commercial production of palm oil.

1. A method for predicting palm oil yield of a test oil palm plant, the method comprising the steps of:
(i) determining, from a sample of a test oil palm plant of a population of oil palm plants, at least a first single nucleotide polymorphism (SNP) genotype of the test oil palm plant, the first SNP genotype corresponding to a first SNP marker, the first SNP marker (a) being located in a first quantitative trait locus (QTL) for a high-oil-production trait and (b) being associated, after stratification and kinship correction, with the high-oil-production trait with a genome-wide −log₁₀(p-value) of at least 7.0 in the population or having a linkage disequilibrium r² value of at least 0.2 with respect to a first other SNP marker that is linked thereto and associated, after stratification and kinship correction, with the high-oil-production trait with a genome-wide −log₁₀(p-value) of at least 7.0 in the population;
(ii) comparing the first SNP genotype of the test oil palm plant to a corresponding first reference SNP genotype indicative of the high-oil-production trait in the same genetic background as the population; and
(iii) predicting palm oil yield of the test oil palm plant based on the extent to which the first SNP genotype of the test oil palm plant matches the corresponding first reference SNP genotype,
wherein the first QTL is a region of the oil palm genome corresponding to one of:
(1) QTL region 1, extending from nucleotide 1516571 to 4215826 of chromosome 2;
(2) QTL region 2, extending from nucleotide 4858549 to 5594262 of chromosome 2;
(3) QTL region 3, extending from nucleotide 33949264 to 34110104 of chromosome 2;
(4) QTL region 4, extending from nucleotide 43405853 to 43834266 of chromosome 3;
(5) QTL region 5, extending from nucleotide 44126148 to 44193097 of chromosome 3;
(6) QTL region 6, extending from nucleotide 30702027 to 31148630 of chromosome 4;
(7) QTL region 7, extending from nucleotide 33166529 to 33451554 of chromosome 4;
(8) QTL region 8, extending from nucleotide 35906266 to 36257708 of chromosome 7;
(9) QTL region 9, extending from nucleotide 29233675 to 29612202 of chromosome 10;
(10) QTL region 10, extending from nucleotide 13470988 to 13734716 of chromosome 11;
(11) QTL region 11, extending from nucleotide 24620951 to 24989005 of chromosome 13; or
(12) QTL region 12, extending from nucleotide 6941783 to 7160542 of chromosome 15.

2. The method of claim 1, wherein the high-oil-production trait comprises decreased shell-to-fruit, increased mesocarp-to-fruit, or a combination thereof, in tenera oil palm plants.

3. The method of claim 2, wherein the high-oil-production trait comprises decreased shell-to-fruit in tenera oil palm plants.

4. The method of claim 2, wherein the high-oil-production trait comprises increased mesocarp-to-fruit in tenera oil palm plants.

5. The method of claim 2, wherein the high-oil-production trait comprises decreased shell-to-fruit and increased mesocarp-to-fruit in tenera oil palm plants.

6. The method of claim 1 or 2, wherein:
the first QTL corresponds to one of QTL regions 1, 2, 3, 4, 5, 6, 7, or 10;
the high-oil-production trait comprises decreased shell-to-fruit; and
step (iii) further comprises applying a genotype model, thereby predicting the palm oil yield of the test oil palm plant.

7. The method of claim 1 or 2, wherein:
the first QTL corresponds to one of QTL regions 1, 8, 9, 11, or 12;
the high-oil-production trait comprises increased mesocarp-to-fruit; and
step (iii) further comprises applying a genotype model, thereby predicting the palm oil yield of the test oil palm plant.

8. The method of claim 1 or 2, wherein:
the first QTL corresponds to QTL region 1;
the high-oil-production trait comprises decreased shell-to-fruit and increased mesocarp-to-fruit; and
step (iii) further comprises applying a genotype model, thereby predicting the palm oil yield of the test oil palm plant.

9. The method of claim 1 or 2, wherein:
the first QTL corresponds to QTL region 1;
the high-oil-production trait comprises decreased shell-to-fruit; and
step (iii) further comprises applying a dominant model, thereby predicting the palm oil yield of the test oil palm plant.

10. The method of claim 1 or 2, wherein:
the first QTL corresponds to QTL region 1;
the high-oil-production trait comprises decreased shell-to-fruit; and
step (iii) further comprises applying a recessive model, thereby predicting the palm oil yield of the test oil palm plant.

11. The method of claim 1 or 2, wherein:
the first QTL corresponds to QTL region 1;
the high-oil-production trait comprises increased mesocarp-to-fruit; and
step (iii) further comprises applying a recessive model, thereby predicting the palm oil yield of the test oil palm plant.

12. The method of claim 1 or 2, wherein:
the first QTL corresponds to QTL region 1;
the high-oil-production trait comprises decreased shell-to-fruit and increased mesocarp-to-fruit; and
step (iii) further comprises applying a recessive model, thereby predicting the palm oil yield of the test oil palm plant.

13. The method of any one of claims 1-12, wherein the test oil palm plant is a tenera candidate agricultural production plant.

14. The method of claim 1 or 2, wherein the test oil palm plant is a plant for mother palm selection and propagation, a plant for introgressed mother palm selection and propagation, or a plant for pollen donor selection and propagation.

15. The method of any one of claims 1-14, wherein the test oil palm plant is a seed, a seedling, a nursery phase plant, an immature phase plant, a cell culture plant, a zygotic embryo culture plant, or a somatic tissue culture plant.

16. The method of any one of claims 1-14, wherein the test oil palm plant is a production phase plant, a mature palm, a mature mother palm, or a mature pollen donor.

17. The method of any one of claims 1-16, wherein:
step (i) further comprises determining, from the sample of the test oil palm plant, at least a second SNP genotype of the test oil palm plant, the second SNP genotype corresponding to a second SNP marker, the second SNP marker (a) being located in a second QTL for the high-oil-production trait and (b) being associated, after stratification and kinship correction, with the high-oil-production trait with a genome-wide −log₁₀(p-value) of at least 7.0 in the population or having a linkage disequilibrium r² value of at least 0.2 with respect to a second other SNP marker that is linked thereto and associated, after stratification and kinship correction, with the high-oil-production trait with a genome-wide −log₁₀(p-value) of at least 7.0 in the population; and
step (ii) further comprises comparing the second SNP genotype of the test oil palm plant to a corresponding second reference SNP genotype indicative of the high-oil-production trait in the same genetic background as the population,
wherein the second QTL corresponds to one of QTL regions 1 to 12, with the proviso that the first QTL and the second QTL correspond to different QTL regions.

18. The method of claim 17, wherein step (iii) further comprises predicting palm oil yield of the test oil palm plant based on the extent to which the second SNP genotype of the test oil palm plant matches the corresponding second reference SNP genotype.

19. The method of claim 17 or 18, wherein:
step (i) further comprises determining, from the sample of the test oil palm plant, at least a third SNP genotype to a twelfth SNP genotype of the test oil palm plant, the third SNP genotype to the twelfth SNP genotype corresponding to a third SNP marker to a twelfth SNP marker, respectively, the third SNP marker to the twelfth SNP marker (a) being located in a third QTL to a twelfth QTL, respectively, for the high-oil-production trait and (b) being associated, after stratification and kinship correction, with the high-oil-production trait with a genome-wide −log₁₀(p-value) of at least 7.0 in the population or having linkage disequilibrium r² values of at least 0.2 with respect to a third other SNP marker to a twelfth other SNP marker, respectively, that are linked thereto and associated, after stratification and kinship correction, with the high-oil-production trait with a genome-wide −log₁₀(p-value) of at least 7.0 in the population; and
step (ii) further comprises comparing the third SNP genotype to the twelfth SNP genotype of the test oil palm plant to a corresponding third reference SNP genotype to a corresponding twelfth reference SNP genotype, respectively, indicative of the high-oil-production trait in the same genetic background as the population,
wherein the third QTL to the twelfth QTL each correspond to one of QTL regions 1 to 12, with the proviso that the first QTL to the twelfth QTL each correspond to different QTL regions.

20. The method of claim 19, wherein step (iii) further comprises predicting palm oil yield of the test oil palm plant based on the extent to which the third SNP genotype to the twelfth SNP genotype of the test oil palm plant match the corresponding third reference SNP genotype to the corresponding twelfth reference SNP genotype, respectively.

21. A method of selecting a high-palm-oil-yielding oil palm plant for agricultural production of palm oil, the method comprising the steps of:
(a) predicting palm oil yield of a test oil palm plant according to the method of any one of claims 1-20; and
(b) field planting the test oil palm plant for agricultural production of palm oil if the palm oil yield of the test oil palm plant is predicted to be higher than average for the population based on step (a).

22. A method of selecting a high-palm-oil-yielding oil palm plant for cultivation in cell culture, the method comprising the steps of:
(a) predicting palm oil yield of a test oil palm plant according to the method of any one of claims 1-20; and
(b) subjecting at least one cell of the test oil palm plant to cultivation in cell culture if the palm oil yield of the test oil palm plant is predicted to be higher than average for the population based on step (a).

23. A method of selecting a parental oil palm plant for use in breeding to obtain agricultural production plants or improved parental oil palm plants, the method comprising the steps of:
(a) predicting palm oil yield of a test oil palm plant according to the method of any one of claims 1-20; and
(b) selecting the test oil palm plant for use in breeding if the palm oil yield of tenera progeny of the test oil palm plant is predicted to be higher than average for the population based on step (a).
